(19) United States
(12) Patent Application Publication
(10) Pub. No.: US 2025/0090462 A1
(43) Pub. Date: Mar. 20, 2025

(54) LIPID COMPOUND AND PREPARATION METHOD THEREFOR, AND USE THEREOF

(71) Applicant: South China University of Technology, Guangzhou (CN)

(72) Inventors: Yuan ZHANG, Guangzhou (CN); Zhengqiang FU, Guangzhou (CN)

(73) Assignee: South China University of Technology, Guangzhou (CN)

(21) Appl. No.: 18/970,983

(22) Filed: Dec. 6, 2024

Related U.S. Application Data
(63) Continuation of application No. PCT/CN2023/098778, filed on Jun. 7, 2023.

(30) Foreign Application Priority Data
Jun. 10, 2022 (CN) 202210653708.1

(51) Int. Cl. A61K 9/1271; A61K 9/51; A61K 45/06; C07C 271/16; C07D 207/06; C07D 207/09; C07D 211/14; C07D 233/61; C07D 295/13

(52) U.S. Cl. CPC A61K 9/1271; A61K 9/5123; A61K 45/06; C07C 271/16; C07D 207/06; C07D 207/09; C07D 211/14; C07D 233/61; C07D 295/13

(57) ABSTRACT

A lipid compound and a preparation method therefor, and a use thereof are provided. The lipid compound is safe, efficient and ionizable and the structure of the lipid compound is divided into a hydrophilic amino group, a linking group, and a hydrophobic alkyl group. The preparation method of the lipid compound is simple, green and efficient. The lipid compound can be widely used in preparation of a drug carrier.

### CROSS REFERENCE TO THE RELATED APPLICATIONS

This application is a continuation application of International Application No. PCT/CN2023/098778, filed on Jun. 7, 2023, which is based upon and claims priority to Chinese Patent Application No. 202210653708.1, filed on Jun. 10, 2022, the entire contents of which are incorporated herein by reference.

### TECHNICAL FIELD

The invention belongs to the pharmaceutical field, and specifically relates to a lipid compound and a preparation method therefor, and a use thereof.

### BACKGROUND

The representative of gene therapy is a class of nucleic acid drugs such as small interfering RNA (siRNA), message RNA (mRNA), plasmid DNA (pDNA), and other exogenous genes with therapeutic purposes are delivered to target cells through carrier materials to exert therapeutic effects. The mRNA vaccine has become a promising platform for cancer immunotherapy. During vaccination, naked or drug-loaded mRNA vaccines effectively express tumor antigens in antigen presenting cells (APCs), promoting APC activation and innate/adaptive immune stimulation. However, mRNA therapy still faces the challenge of lacking a safe and effective delivery system, because the large size and dense negative charge make it difficult for bare mRNA to pass through the cell membrane. In addition, mRNA itself is also an unstable molecule that is easily degraded. Therefore, developing an efficient and safe nucleic acid delivery system is the cornerstone of gene therapy.

At present, the most common vectors in nucleic acid delivery systems are two types of vectors: viral vectors and non-viral vectors, the transfection efficiency of viral vectors is relatively high, but there are problems such as poor safety and targeting. Over the past decades, non-viral vectors represented by liposomes have developed rapidly. Because of their low immunogenicity, good biocompatibility, and high transfection efficiency, they are regarded as ideal nucleic acid delivery systems. Compared with traditional liposomes, the stability and transfection efficiency of ionizable lipids in vivo are greatly improved, and they are electrically neutral during transportation in vivo, resulting in low biological toxicity. An ionizable lipid is an amphiphilic structure with a hydrophilic head, which contains one or more ionizable amines and multiple hydrophobic alkane chains that promote self-assembly, as well as a linker that connects the head and tail. Ionizable lipid protonates the amine head at acidic pH to obtain a positive charge, which can promote the binding of positively charged lipids to negatively charged mRNA through electrostatic interaction. When lipid nanoparticles (LNP) are transported in the intracellular environment, the acidic microenvironment can interact with the positively charged lipid and the ionic inner membrane, promoting membrane fusion and instability, thereby releasing mRNA from LNPs into the cytoplasm.

The existing RNA therapy is very sensitive to nucleases because of the RNA it loads, and RNA is large, negatively charged, and unable to penetrate the cell membrane. Existing technologies can transfer RNA to target cells through lipid nanoparticles, providing a huge cure for a series of diseases including COVID-19. However, the traditional lipid compound nucleic acid delivery system has problems such as low efficiency, high toxicity, and poor targeting. Therefore, it is necessary to develop a lipid compound with high efficiency, low toxicity, and excellent targeting.

### SUMMARY

In order to overcome the problems of low efficiency, high toxicity, and poor targeting of lipid compounds in existing technologies, one of the purposes of the invention is to provide a lipid compound; the second purpose of the invention is to provide a preparation method for the lipid compound; the third purpose of the invention is to provide a use of the lipid compound; the fourth purpose of the invention is to provide a drug composition.

In order to achieve the above purpose, the technical scheme adopted by the invention is as follows:
The first aspect of the invention provides a lipid compound, and a structure of the lipid compound is shown in Formula (I);

A-(B)ₙ (I);

In Formula (I), A is selected from a structure shown in Formula (1)-Formula (18), B is selected from a structure shown in Formula (19), and n is selected from a positive integer of 2-4;

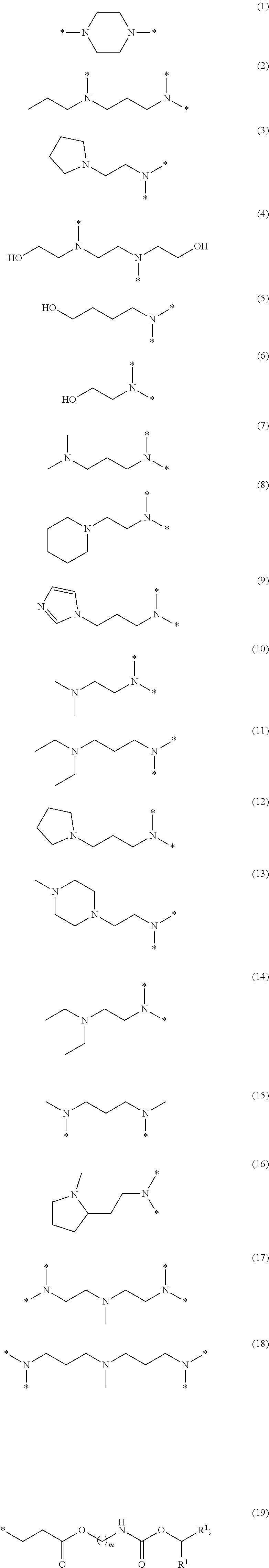

In Formula (19), R¹ is selected from an alkyl or an alkenyl group of C6-C12, and m is selected from the positive integers of 1-6.

Preferably, in Formula (I), A is selected from the structure shown in Formula (3), Formula (5)-Formula (16), B is selected from the structure shown in Formula (19), n is 2;
in Formula (19), R¹ is selected from the alkyl group of C7-C9, and m is selected from the positive integers of 2-5.

Preferably, the lipid compound includes a compound with the following structures:

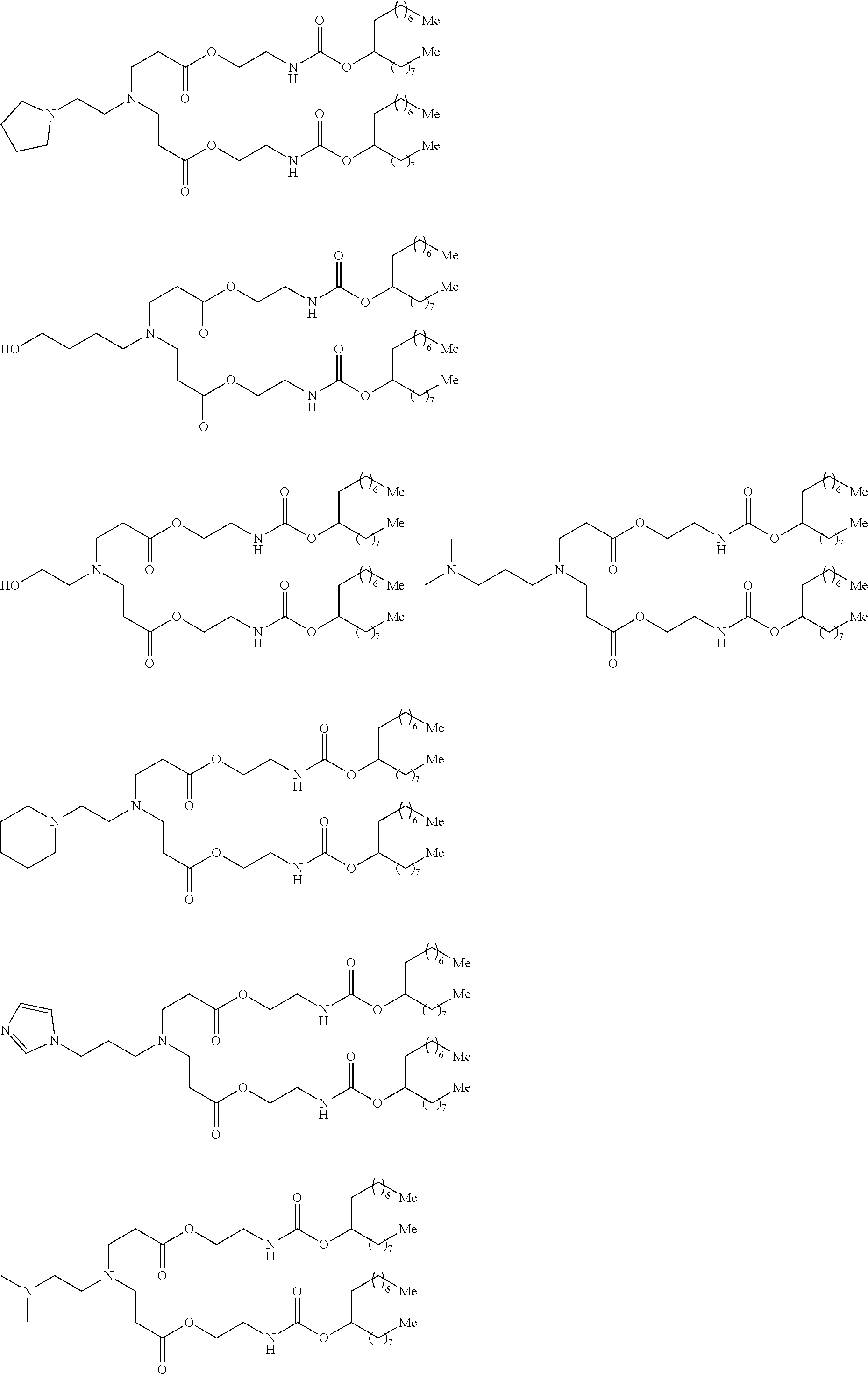

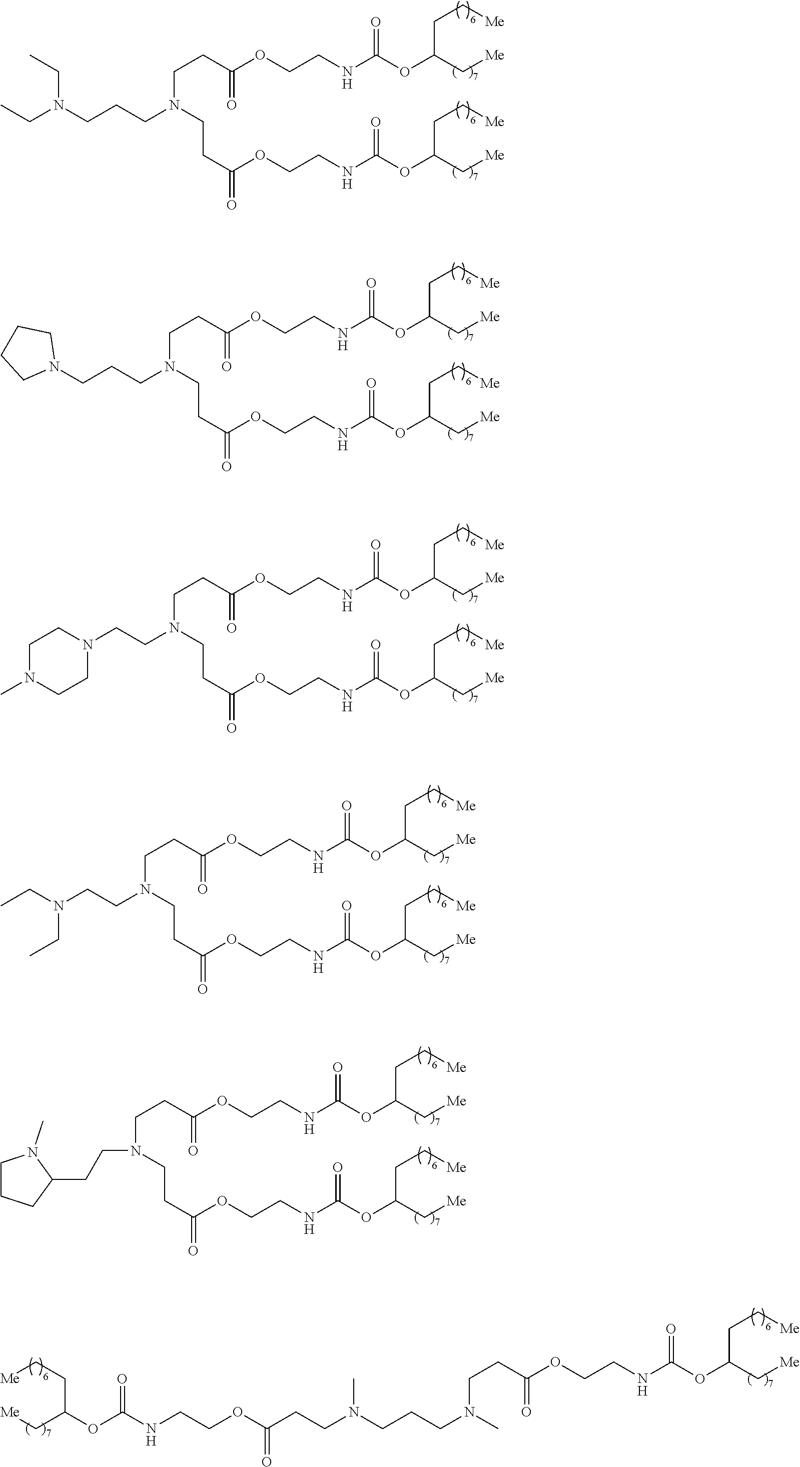

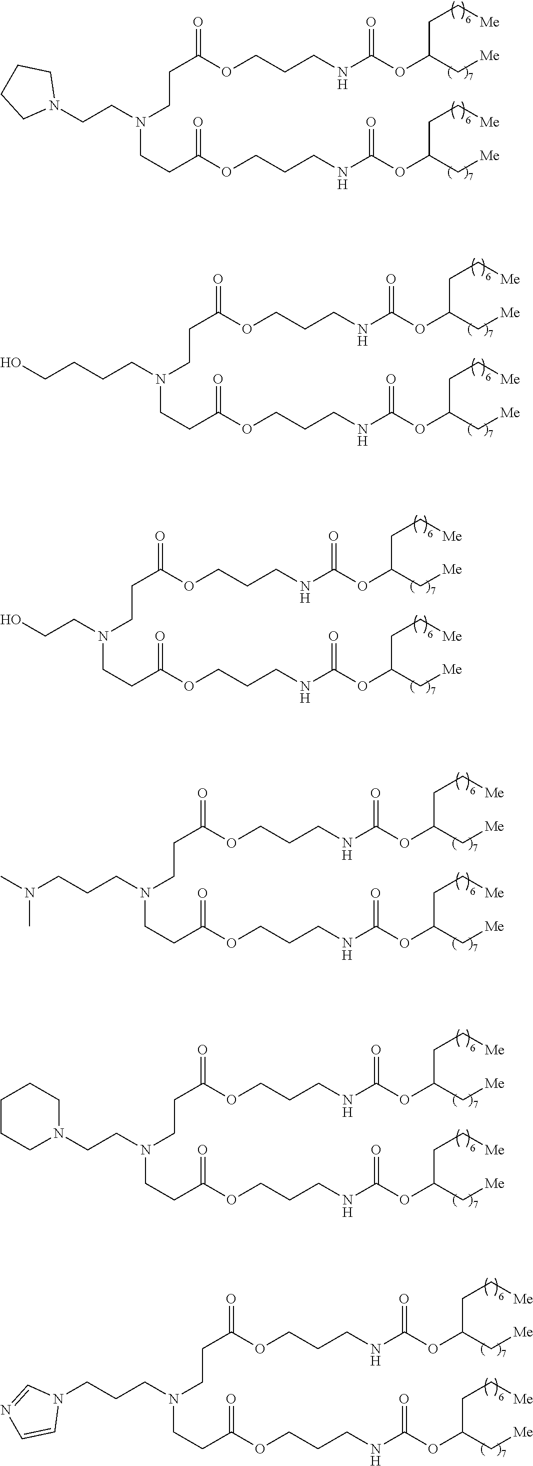

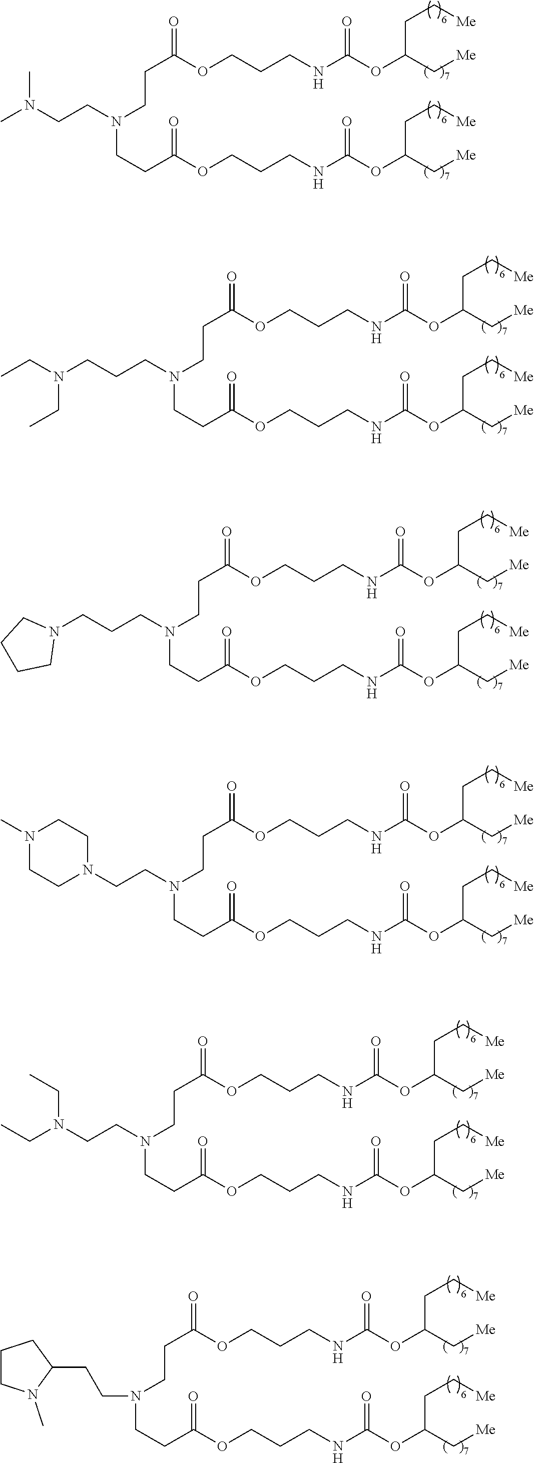

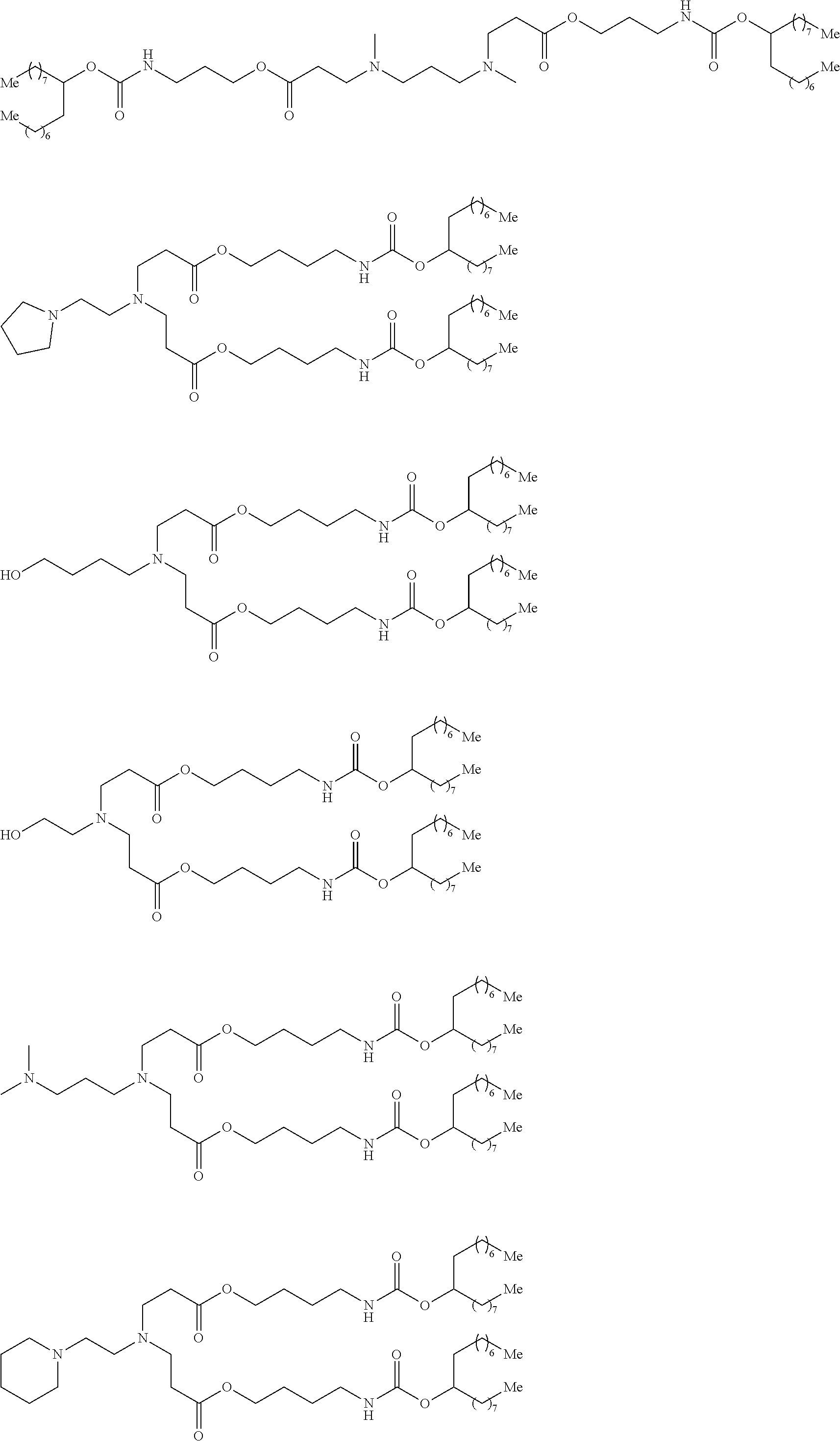

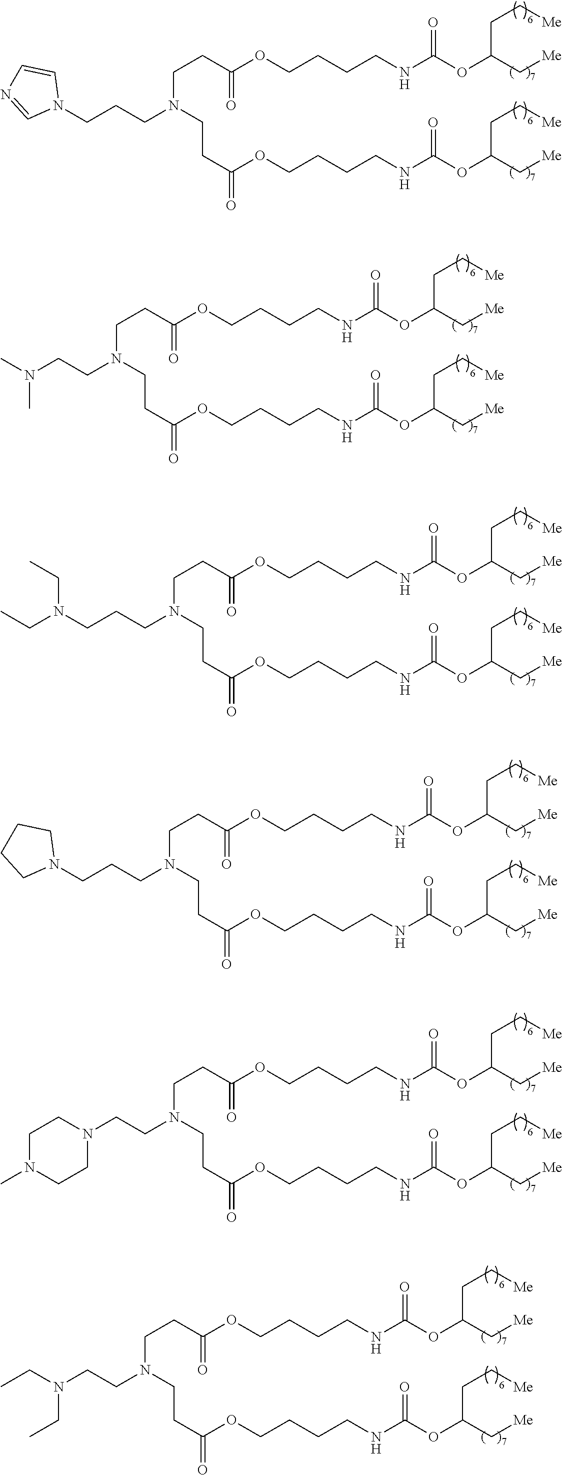

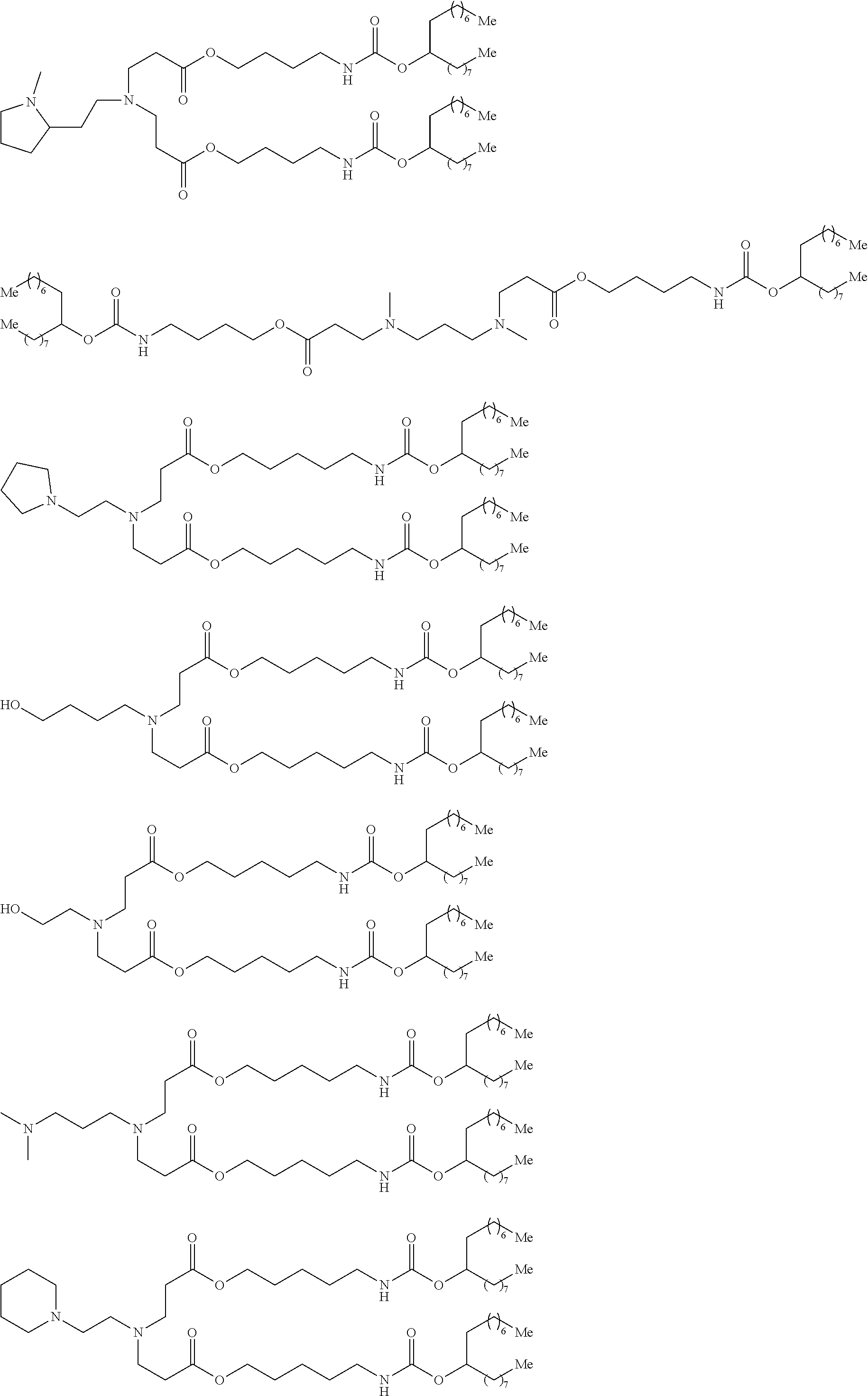

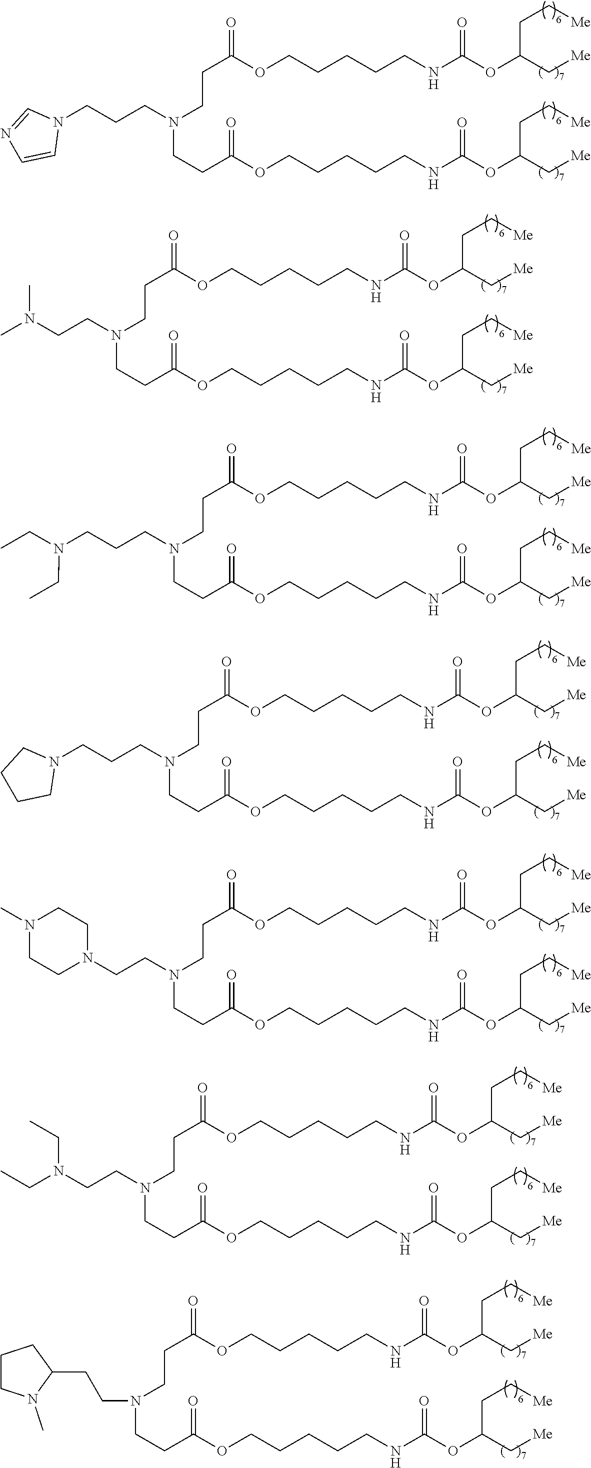

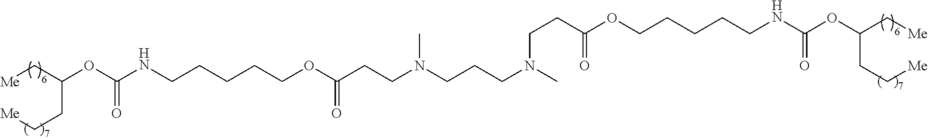

The second aspect of the invention provides a preparation method for the lipid compound according to the first aspect of the invention, including the following steps:

Mixing the compound shown in Formula (II) with the compound shown in Formula (20)-Formula (37), reacting, and obtaining the lipid compound.

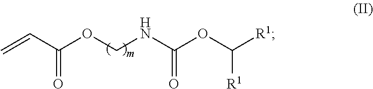

In Formula (II), R¹ is selected from the alkyl or the alkenyl group of C6-C12, m is selected from the positive integers of 1-6;

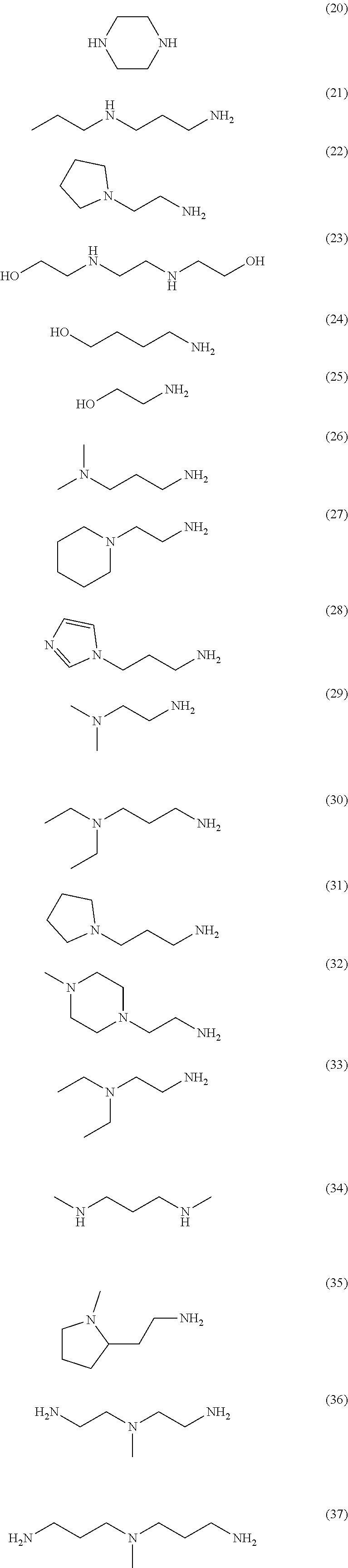

Preferably, a molar ratio of an active hydrogen atom in the compound shown in Formula (II) to the compound selected from Formula (20)-Formula (37) is (1-2):1; furthermore, the molar ratio of the active hydrogen atom in the compound shown in Formula (II) to the compound selected from Formula (20)-Formula (37) is (1-1.5):1.

Preferably, the active hydrogen atom is a hydrogen atom connected with a nitrogen atom.

Preferably, a reaction time is 24 h-72 h; furthermore, the reaction time is 36 h-60 h.

Preferably, a reaction temperature is 70° C.-110° C.; furthermore, the reaction temperature is 80° C.-100° C.

Preferably, a reaction is a solvent-free reaction.

The third aspect of the invention provides a use of the lipid compound according to the first aspect of the invention in a preparation of a nucleic acid drug, a gene vaccine, a polypeptide or protein drug, and a small molecule drug.

Preferably, a nitrogen-phosphorus ratio of the nucleic acid drug is (12-36):1; furthermore, the nitrogen-phosphorus ratio of the nucleic acid drug is (18-30):1.

The fourth aspect of the invention provides a use of the lipid compound according to the first aspect of the invention in a preparation of a drug carrier.

Preferably, drugs include an RNA drug, a DNA drug, a polypeptide or protein drug, or a small molecule drug;

Preferably, the RNA drug includes at least one of siRNA, microRNA (miRNA), mRNA, synthetic chemically modified RNA (modRNA), circular RNA (circRNA), antisense RNA, clustered regularly interspaced short palindromic repeats (CRISPR) guide RNAs, self-replicating RNA (repRNA), cyclic dinucleotide (CDN), poly IC, CpG oligonucleotide (ODN), pDNA, and microcyclic DNA.

Preferably, the DNA drug includes a plasmid DNA.

Preferably, the protein drug includes at least one of a cell colony stimulating factor, interleukin, lymphotoxin, interferon, a tumor necrosis factor, antibody, and protein antigen.

The fifth aspect of the invention provides a drug composition, the drug composition includes the lipid compound described above, or its pharmaceutically acceptable salt or its stereoisomer. Furthermore, the drug composition also includes at least one of cholesterol and cholesterol derivative, helper phospholipid, and polyethylene glycol modified lipid.

The helper phospholipid includes at least one of egg yolk lecithin, hydrogenated egg yolk lecithin, soybean lecithin, hydrogenated soybean lecithin, sphingomyelin, phosphatidylethanolamine, dimyristoylphosphatidylcholine, dimyristoylphosphatidylglycerol, dipalmitoylphosphatidylcholine, distearoylphosphatidylcholine (DSPC), dioleoyl phosphatidyl ethanolamine (DOPE), dioleoylphosphatidylcholine (DOPC), dioleoylphosphatidylcholine and dilauroylphosphatidylcholine. Preferably, the polyethylene glycol (PEG) modified lipid includes at least one of PEG modified phosphatidylethanolamine, PEG modified phosphatidic acid, PEG modified ceramide, PEG modified dialkylamine, PEG modified diacylglycerol, PEG modified dialkylglycerol, ALC-0159 and PEG modified products of the above compounds (e.g. -Maleimide, —COOH, —NH₂).

Preferably, when the drug composition also includes at least one of cholesterol and cholesterol derivative, helper phospholipid and polyethylene glycol modified lipid, a molar ratio of the lipid compound, or its pharmaceutically acceptable salt or its stereoisomers:cholesterol and cholesterol derivative:helper phospholipid:polyethylene glycol modified lipid is (30-50):(30-50):(5-20):(1-2.5).

Preferably, the drug composition also includes an active pharmaceutical ingredient; the active pharmaceutical ingredient includes nucleic acid molecules and a protein drug, the nucleic acid molecules include small interfering RNA (siRNA), microRNA (miRNA), messenger RNA (mRNA), chemically modified mRNA (modRNA), circular RNA (circRNA), antisense RNA, CRISPR guide RNA, self-replicating RNA (repRNA), cyclic dinucleotide (CDN), polyIC, CpG ODN, plasmid DNA (pDNA), and microcyclic DNA; the protein drug includes cell colony stimulating factor, interleukin, lymphotoxin, interferon protein, tumor necrosis factor, antibody, and protein antigen.

Preferably, when the active pharmaceutical ingredient includes nucleic acid molecules, a ratio of the nitrogen in the lipid compound or its pharmaceutically acceptable salt or its stereoisomer to the phosphorus in the nucleic acid molecule is (4-32):1.

Preferably, the drug composition includes a combination of the lipid compound according to the first aspect of the invention with dioleoyl phosphatidyl ethanolamine (DOPE), distearoylphosphatidylcholine (DSPC), cholesterol, and distearoylphosphatidylacetamide-polyethylene glycol (DSPE-PEG).

Preferably, the molar ratio of the lipid compound to dioleoyl phosphatidyl ethanolamine (DOPE) or distearoylphosphatidylcholine (DSPC), cholesterol, and distearoylphosphatidylacetamide-polyethylene glycol (DSPE-PEG) is (10-100):(0-90):(0-90):(0-90); furthermore, the molar ratio of the lipid compound to dioleoyl phosphatidyl ethanolamine (DOPE) or distearoylphosphatidylcholine (DSPC), cholesterol, and distearoylphosphatidylacetamide-polyethylene glycol (DSPE-PEG) is (30-50):(5-15):(40-50):(0-5).

The beneficial effects of the invention are as follows:

The lipid compound provided by the invention is safe, efficient, and ionizable, its structure is divided into a hydrophilic amine group, a connecting group, and a hydrophobic alkyl group, the preparation method of the lipid compound is simple, green, and efficient. The lipid compound can be widely used in the preparation of drug carriers.

Specifically, the invention has the following advantages:
1. The invention provides a lipid compound structure containing an ester bond, which can be rapidly hydrolyzed by enzymes in the body, is easy to metabolize and remove, and is biodegradable; the structure of the lipid compound has a branched chain shape, after assembling the lipid nanoparticles, the cross-sectional area of the hydrophobic part of the lipid can be increased, which helps the nano-drug escape from the endosome, thereby enhancing the transfection effect; the lipid compound can obtain hydrogen protons under acidic conditions and ionize into cations, it can bind to negatively charged nucleic acid molecules through electrostatic interaction, and form lipid nanoparticles with helper lipids, which can effectively deliver mRNA and pDNA into cells and express target genes; the charge of the lipid compound can change with the change of pH, and is electrically neutral under neutral conditions, reducing the cytotoxicity caused by excessive positive charge, thereby increasing the stability of lipid nanoparticles, and rapid degradation is avoided in the body when there is excessive positive charge, it helps to prolong the circulation time of the loaded nucleic acid drug and improve its pharmacokinetic characteristics.
2. Compared with traditional cationic lipids, the ionizable lipid compounds prepared by the invention have the advantages of simple synthesis steps, convenient product separation, high efficiency, and low toxicity. The initial raw materials of lipid compounds prepared by the invention are common and easy to obtain, the reaction conditions are simple and mild, and the synthetic route design is reasonable. The invention is based on common alkyl alcohol compounds and organic amine compounds, ionizable lipids with different ionization groups and different branched chain lengths are synthesized by simple esterification reaction and Michael addition.
3. The lipid compound provided by the invention solves the problem of nucleic acid delivery and can efficiently transfect mRNA in vivo and in vitro, the transfection effect is comparable to that of commercial transfection reagents, and the lipid compound can be widely used in the preparation of drugs and drug carriers.

### BRIEF DESCRIPTION OF THE DRAWINGS

FIG. 1 is a nuclear magnetic hydrogen spectrum of intermediate product B.

FIG. 2 is a nuclear magnetic hydrogen spectrum of intermediate product C.

FIG. 3 is a nuclear magnetic hydrogen spectrum of lipid 3-2-C8 prepared by Embodiment 1.

FIG. 4 is a nuclear magnetic hydrogen spectrum of lipid 10-2-C8 prepared by Embodiment 2.

FIG. 5 is a nuclear magnetic hydrogen spectrum of intermediate product D.

FIG. 6 is a nuclear magnetic hydrogen spectrum of intermediate product E.

FIG. 7 is a nuclear magnetic hydrogen spectrum of lipid 3-4-C8 prepared by Embodiment 3.

FIG. 8 is a nuclear magnetic hydrogen spectrum of lipid 10-4-C8 prepared by Embodiment 4.

FIG. 9 is a relative luciferase activity of the lipid compound.

FIG. 10 is a heat map of the relative luciferase activity of the lipid compound.

FIG. 11 is a particle size test diagram of nanoparticles of the lipid compound.

FIG. 12 is a relative luciferase activity of the lipid compound.

FIG. 13 is a relative luciferase activity of the lipid compound.

FIG. 14 is a test result of the relative luciferase activity of the lipid compound.

FIG. 15 is a fluorescence microscope result of the lipid compound.

FIG. 16 is a relative luciferase activity % of the lipid compound.

FIG. 17 is a distribution map of the lipid nanoparticles in various organs of mice.

FIG. 18 is a detection map of the lipid nanoparticles in vivo imaging system in mice.

FIG. 19 is an injection time and total flux of the lipid compound.

### DETAILED DESCRIPTION OF THE EMBODIMENTS

The following provides further illustration of the specific embodiment of this invention with examples, but the implementation and protection of this invention are not limited to these. It should be pointed out that the following processes, if not specified in detail, can be realized or understood by technicians in this field concerning existing technologies. Reagents or instruments not marked with a manufacturer are regarded as conventional products that can be purchased on the market.

The numbering of the lipid compounds in the embodiments of this application means: The structural formula number of structure A-the m value of the structure shown in Formula (19)-the carbon atom number of R¹ of the structure shown in Formula (19), for example, the number ‘3-2-C8’ indicates that in the structure shown in Formula (I), A is selected from the structure shown in Formula (3), B is selected from the structure shown in Formula (19), and m is selected from 2, R¹ is selected from C8 alkyl. Similarly, the difference between ‘3-3-C8’ and ‘3-2-C8’ is only that m is selected from 3, and the other structures are the same.

### Embodiment 1

The preparation steps of the lipid compound in this case are as follows:

1) 10 mmol of 9-heptadecanol, 30 mL of N,N′-carbonyldiimidazole, 20 mmol of triethylamine and 30 mL of dichloromethane were sequentially added to 100 mL of reaction tube containing magnetons, the reaction tube was heated at 40° C. overnight, when the reaction was cooled to room temperature, it was extracted with dichloromethane and saturated salt water, and washed with 1 mol/L HCl (20 mL). The organic layer was collected, dried, and filtered with anhydrous magnesium sulfate, and the solvent was removed by a rotary evaporator, product A can be used for the next reaction without purification. The specific reaction is as follows;

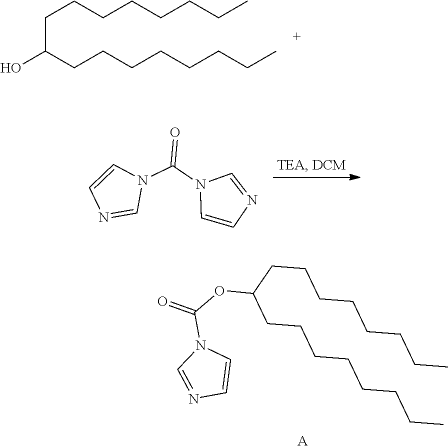

2) 10 mmol of intermediate A, 20 mmol of ethanolamine, and 30 mL of dichloromethane were added to a 100 mL reaction tube containing magnetons, the reaction was heated at 40° C. for 24 hours, and after the reaction was cooled to room temperature, dichloromethane, and saturated salt water were used for extraction. The organic layer was collected, dried, and filtered with anhydrous magnesium sulfate, and the solvent was removed by the rotary evaporator, The product was separated by a thin layer chromatography column (petroleum ether:ethyl acetate volume ratio=5:1) to obtain intermediate product B with a yield of 89%. The specific reaction is as follows;

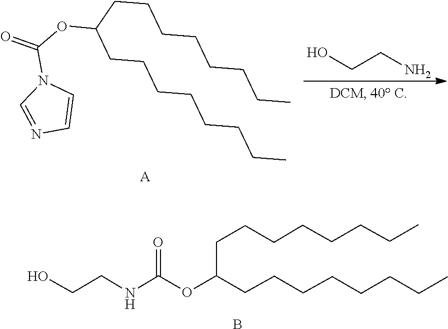

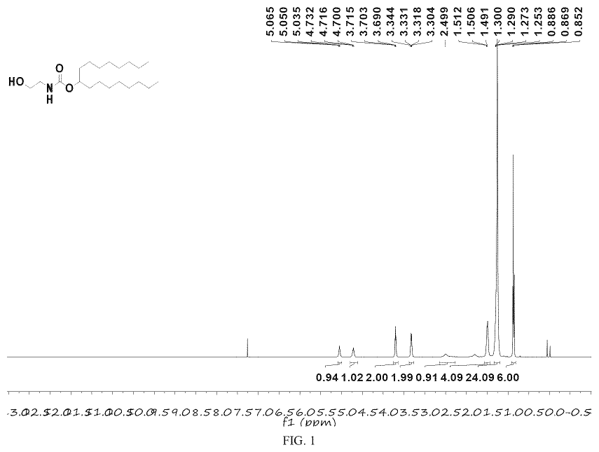

FIG. 1 is the nuclear magnetic hydrogen spectrum of intermediate product B. The specific hydrogen spectrum data are as follows: ¹H NMR (400 MHZ, CDCl₃): 5.07-5.04 (m, 1H), 4.73-4.70 (m, 1H), 3.70 (t, J=4.8 Hz, 2H), 3.34-3.30 (m, 2H), 2.50 (s, 1H), 1.51-1.49 (m, 4H), 1.30-1.25 (m, 24H), 0.87 (t, J=6.8 Hz, 6H).

3) 5 mmol of intermediate B, 7.5 mmol of triethylamine, and 20 mL of dichloromethane were added to a three-port flask containing magnetons, precooled for 30 min under ice bath conditions, and then 6.25 mmol of acryloyl chloride was slowly added by using a constant pressure funnel. After the acryloyl chloride was added, the ice bath was removed, the reaction was carried out overnight at room temperature, and then diluted with dichloromethane (30 mL) and washed with 1 mol/L HCl (50 mL). The organic layer was dried and filtered with anhydrous magnesium sulfate, and the solvent was removed by a rotary evaporator, the product was separated by a thin layer chromatography column (petroleum ether:ethyl acetate=20:1) to obtain intermediate product C with a yield of 81%. The specific reaction is as follows;

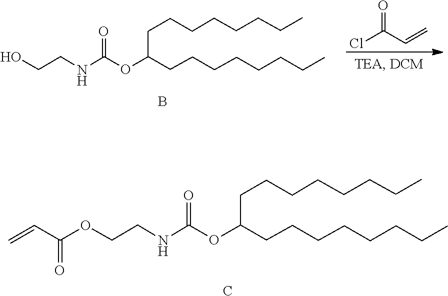

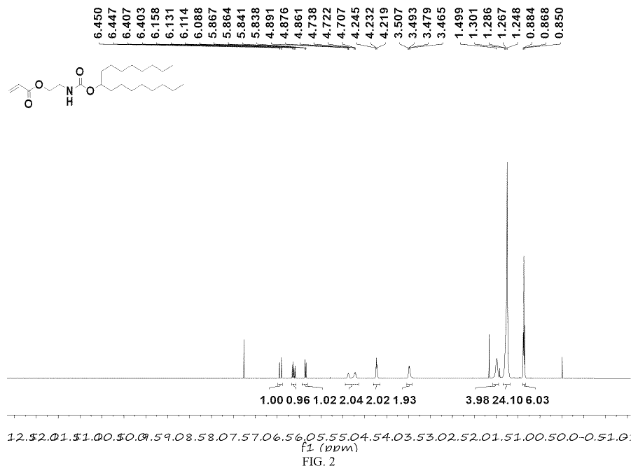

FIG. 2 is the nuclear magnetic hydrogen spectrum of intermediate product C. 1H NMR (400 MHZ, CDCl₃): 6.43 (dd, J=17.2, 1.2 Hz, 1H), 6.12 (q, J=6.8 Hz, 1H), 5.85 (dd, J=10.4, 1.2 Hz, 1H), 4.80 (dt, J=49.2, 6.4 Hz, 2H), 4.23 (t, J=5.2 Hz, 2H), 3.51-3.47 (m, 2H), 1.50 (s, 4H), 1.30-1.25 (m, 24H), 0.87 (t, J=6.4 Hz, 6H).

4) 200 mg of 1-(2-aminoethyl) pyrrolidine and 2 times the chemical equivalent of intermediate product C were added to a 3 mL reaction flask containing magnetons, intermediate product C was stirred at 90° C. for 48 h to obtain the lipid, and the lipid was separated and purified by a thin layer chromatography column (DCM:methanol=20:1) to obtain the target product, the name of the lipid in this embodiment was recorded as 3-2-C8. The specific reaction is as follows;

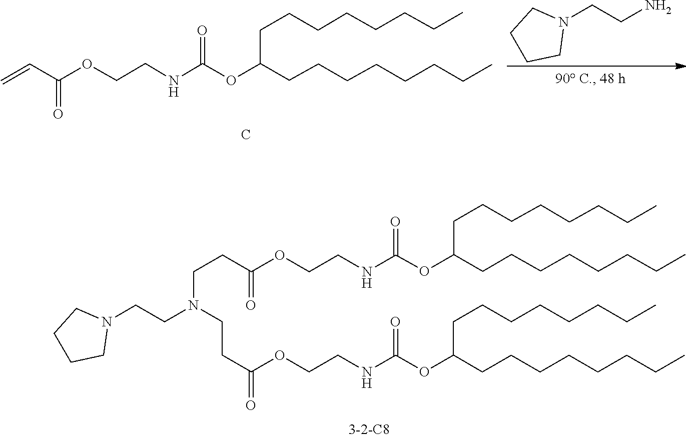

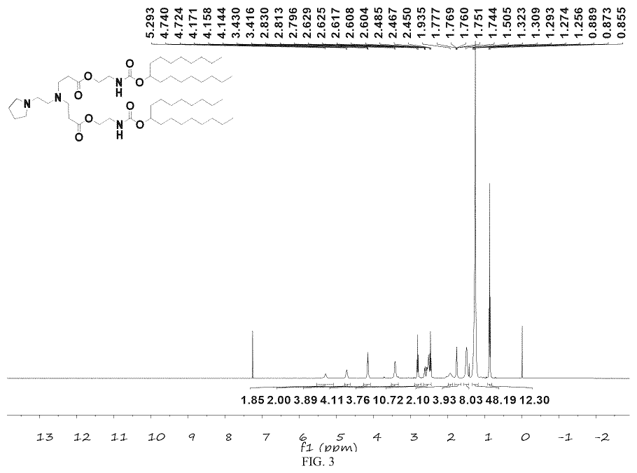

FIG. 3 is the nuclear magnetic hydrogen spectrum of lipid 3-2-C8 prepared by Embodiment 1. ¹H NMR (400 MHZ, CDCl₃): 5.29 (s, 2H), 4.74-4.72 (m, 2H), 4.16 (t, J=5.2 Hz, 4H), 3.43-3.42 (m, 4H), 2.80 (t, J=6.8 Hz, 4H), 2.59-2.35 (m, 8H), 2.22 (s, 6H), 1.53 (s, 8H). 1.29-1.25 (m, 48H), 0.87 (t, J=6.4 Hz, 12H).

### Embodiment 2

The preparation steps of the lipid compound in this embodiment are as follows:

1) The preparation process of intermediate product C is the same as that of Embodiment 1.

2) 200 mg 2-(dimethylamino)ethylamine and 2 times the chemical equivalent of intermediate product C were added to a 3 mL reaction flask containing magnetons, and stirred at 90° C. for 48 h to obtain the lipid, the lipid was separated and purified by a thin layer chromatography column (DCM:methanol=20:1) to obtain the target product, the name of the lipid in this embodiment was recorded as 10-2-C8. The specific reaction is as follows:

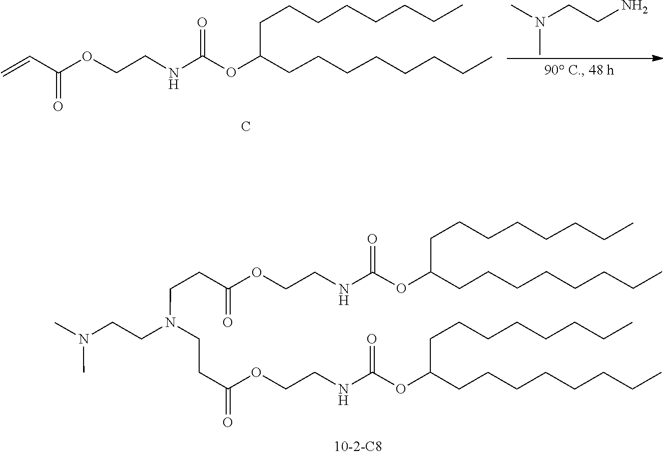

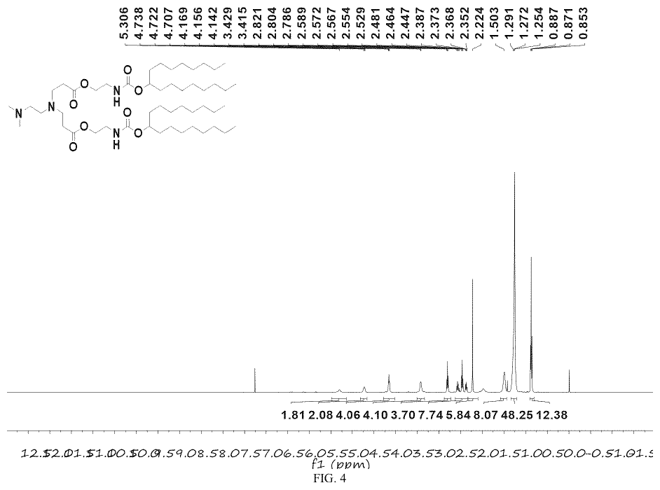

FIG. 4 is the nuclear magnetic hydrogen spectrum of lipid 10-2-C8 prepared by Embodiment 2. ¹H NMR (400 MHZ, CDCl₃): 5.31 (s, 2H), 4.74-4.71 (m, 2H), 4.16 (t, J=5.2 Hz, 4H), 3.43-3.42 (m, 4H), 2.81 (t, J=6.8 Hz, 4H), 2.63-2.45 (m, 10H), 1.94 (s, 2H), 1.78-1.74 (m, 4H). 1.51 (s, 8H), 1.32-1.26 (m, 48H), 0.87 (t, J=6.4 Hz, 12H).

### Embodiment 3

The preparation steps of the lipid compound in this embodiment are as follows:

1) The preparation process of intermediate product A is the same as that in Embodiment 1.

2) 10 mmol of intermediate A, 20 mmol of 4-amino-1-butanol, and 30 mL of dichloromethane were added to a 100 mL reaction tube containing magnetons, the reaction was heated at 40° C. for 24 hours, after the reaction was cooled to room temperature, the solution was extracted with DCM and saturated salt water. The organic layer was dried and filtered with anhydrous magnesium sulfate, and the solvent was removed by a rotary evaporator, the product was separated by a thin layer chromatography column (petroleum ether:ethyl acetate=5:1) to obtain intermediate product D with a yield of 83%. The specific reaction is as follows:

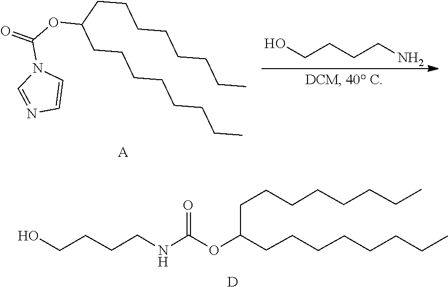

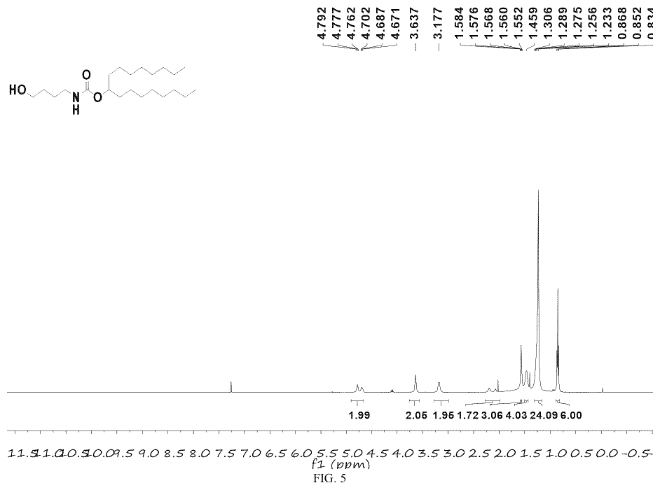

FIG. 5 is the nuclear magnetic hydrogen spectrum of intermediate product D. 1H NMR (400 MHZ, CDCl₃): 4.79-4.67 (m, 2H), 3.64 (s, 2H), 3.18 (s, 2H), 2.20-2.02 (m, 2H), 1.58-1.55 (m, 3H), 1.46 (s, 4H), 1.31-1.23 (m, 24H), 0.85 (t, J=6.4 Hz, 6H).

3) 5 mmol of intermediate product D, 7.5 mmol of triethylamine and 20 mL of DCM were added to a three-necked flask containing magnetons, precooled in an ice bath for 30 min, and then 6.25 mmol of acryloyl chloride was slowly added drop by drop using a constant pressure funnel, after the acryloyl chloride was added, the ice bath was removed, the reaction was carried out overnight at room temperature, and then diluted with DCM (30 mL) and washed with 1 mol/L HCl (50 mL). The organic layer was dried and filtered with anhydrous magnesium sulfate, and the solvent was removed by a rotary evaporator. The product was separated by a thin layer chromatography column (petroleum ether:ethyl acetate=15:1) to obtain intermediate product E with a yield of 80%. The specific reaction is as follows:

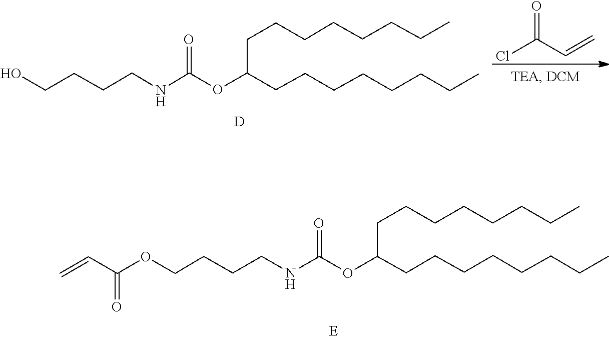

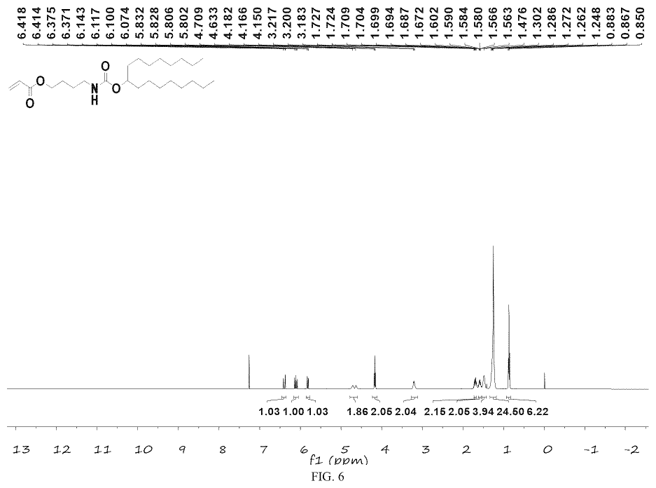

FIG. 6 is the nuclear magnetic hydrogen spectrum of intermediate product E. 1H NMR (400 MHZ, CDCl₃): 6.40 (dd, J=17.2, 1.6 Hz, 1H), 6.11 (q, J=10.4 Hz, 1H), 5.82 (dd, J=10.4, 1.6 Hz, 1H), 4.67 (dt, J=30.8, 6.4 Hz, 2H), 4.17 (t, J=6.4 Hz, 2H). 3.23-3.18 (m, 2H), 1.73-1.67 (m, 2H), 1.60-1.56 (m, 2H), 1.48 (s, 4H), 1.30-1.25 (m, 24H), 0.87 (t, J=6.4 Hz, 6H).

4) 200 mg of 1-(2-aminoethyl) pyrrolidine and 2 times the chemical equivalent of the intermediate product E were added into a 3 mL reaction flask containing magnetons, and stirred at 90° C. for 48 h to obtain the lipid, the lipid was separated and purified by a thin layer chromatography column (DCM:methanol=20:1) to obtain the target product, the name of the lipid in this embodiment was recorded as 3-4-C8. The specific reaction is as follows:

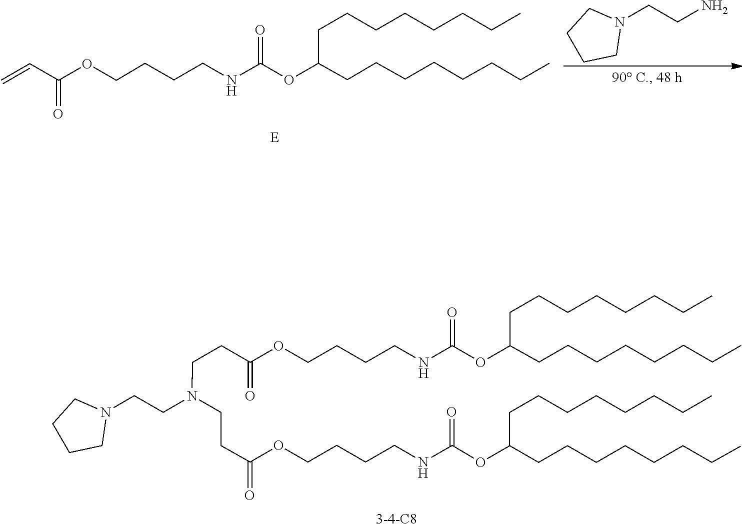

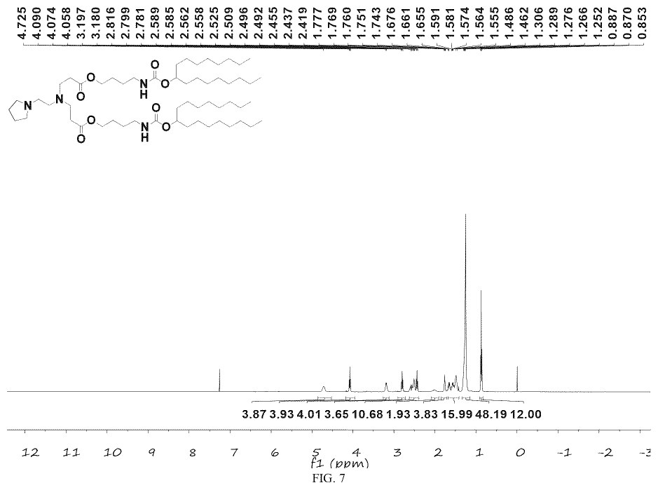

FIG. 7 is the nuclear magnetic hydrogen spectrum of lipid 3-4-C8 prepared by Embodiment 3. ¹H NMR (400 MHZ, CDCl₃): 4.74-4.71 (m, 4H), 4.07 (t, J=6.4 Hz, 4H), 3.20-3.18 (m, 4H), 2.80 (t, J=6.8 Hz, 4H), 2.60-2.42 (m, 10H), 2.04-1.98 (m, 2H), 1.78-1.74 (m, 4H), 1.68-1.46 (m, 16H). 1.31-1.25 (m, 48H), 0.87 (t, J=6.8 Hz, 12H).

### Embodiment 4

The preparation steps of the lipid compound in this embodiment are as follows:

1) The preparation step of intermediate product E is the same as that of Embodiment 3.

2) 200 mg 2-(dimethylamino)ethylamine and 2 times the chemical equivalent of the intermediate product E were added into a 3 mL reaction flask containing magnetons, and stirred at 90° C. for 48 h to obtain the lipid, the lipid was separated and purified by a thin layer chromatography column (DCM:methanol=20:1) to obtain the target product, the name of the lipid in this embodiment was recorded as 10-4-C8. The specific reaction is as follows:

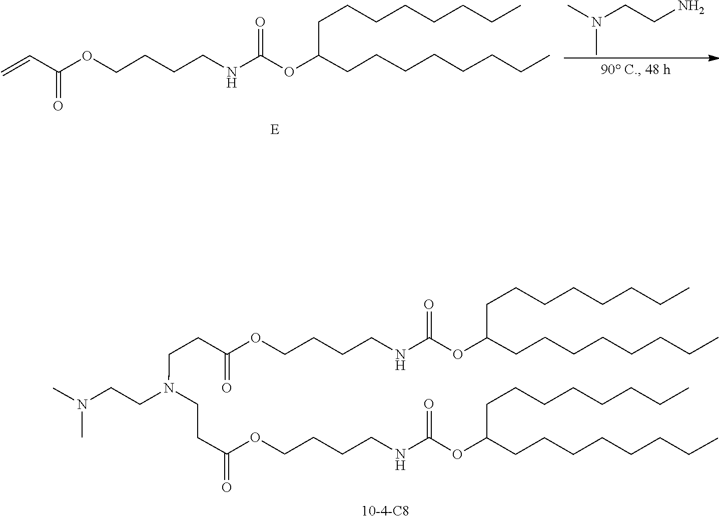

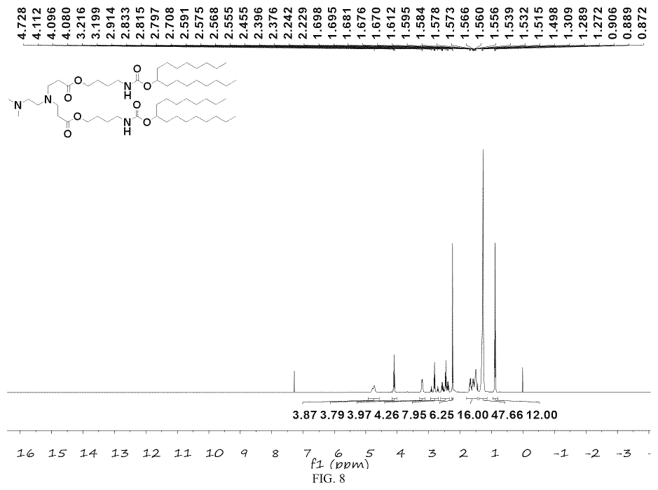

FIG. 8 is the nuclear magnetic hydrogen spectrum of lipid 10-4-C8 prepared by Embodiment 4. ¹H NMR (400 MHZ, CDCl₃): 4.80-4.71 (m, 4H), 4.10 (t, J=6.4 Hz, 4H), 3.22-3.20 (m, 4H), 2.93-2.71 (m, 4H), 2.59-2.38 (m, 8H), 2.24-2.23 (m, 6H), 1.70-1.50 (m, 16H), 1.31-1.27 (m, 48H). 0.89 (t, J=6.8 Hz, 12H).

Concerning the above preparation methods (Michael addition reaction), lipid compounds with other structures can be obtained by using different reaction raw materials, which is not repeated here. Table 1 is the specific structure of the lipid compounds prepared in the embodiments of this invention.

TABLE 1
Specific structure of the lipid compounds prepared in the embodiments of this
invention

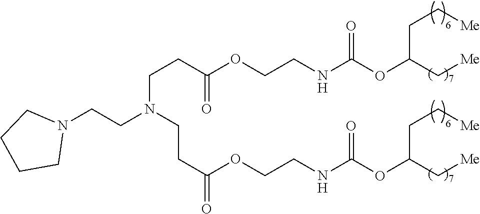

3-2-C8

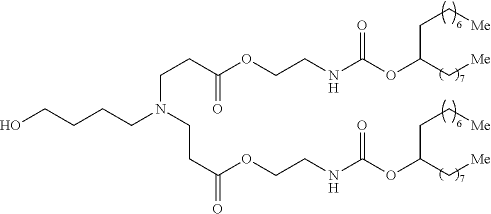

5-2-C8

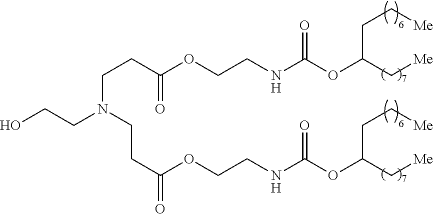

6-2-C8

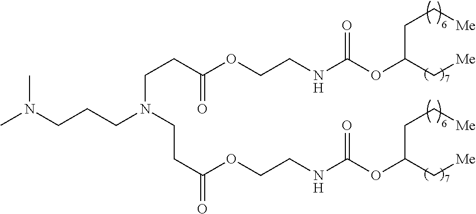

7-2-C8

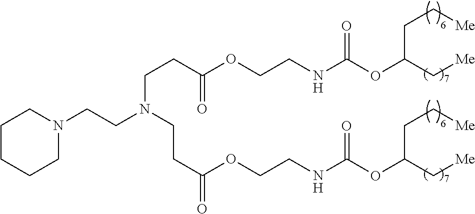

8-2-C8

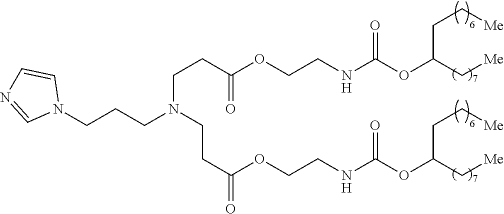

9-2-C8

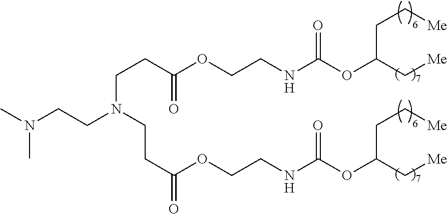

10-2-C8

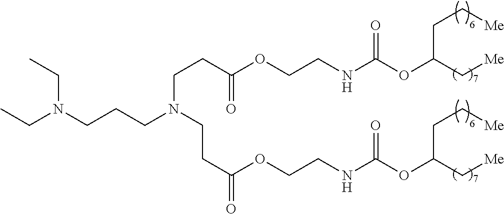

11-2-C8

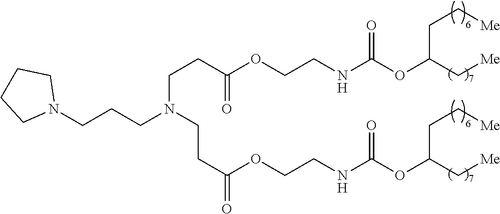

12-2-C8

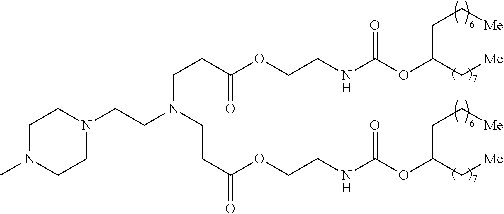

13-2-C8

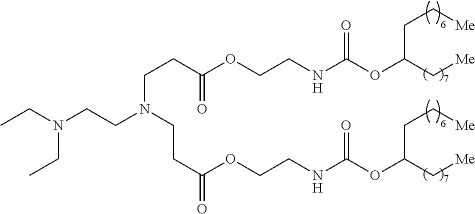

14-2-C8

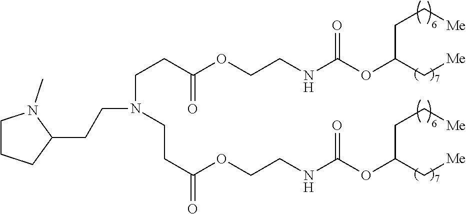

16-2-C8

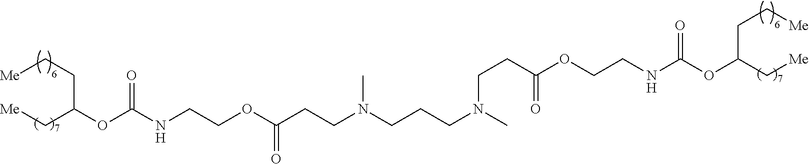

15-2-C8

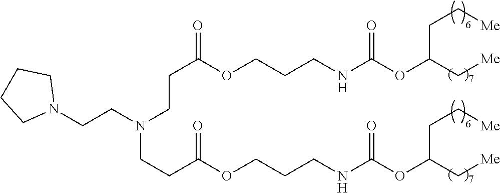

3-3-C8

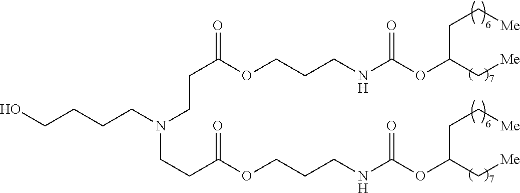

5-3-C8

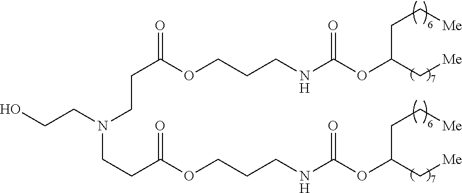

6-3-C8

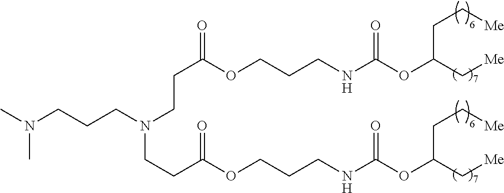

7-3-C8

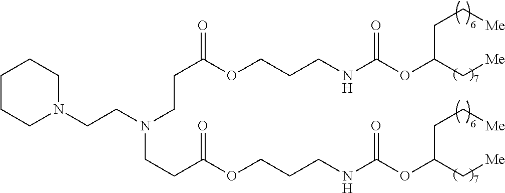

8-3-C8

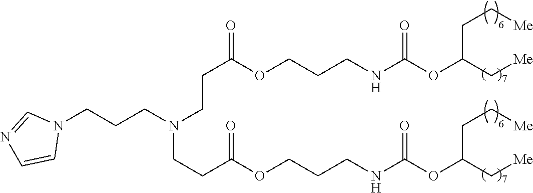

9-3-C8

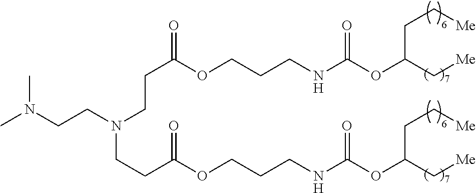

10-3-C8

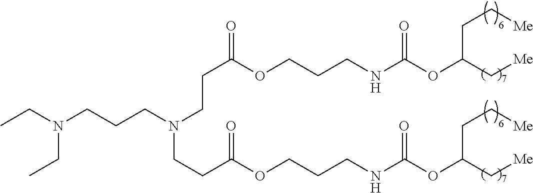

11-3-C8

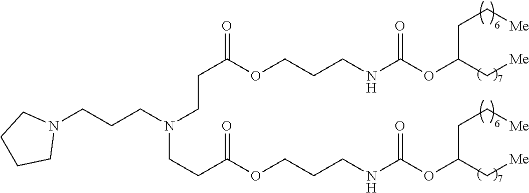

12-3-C8

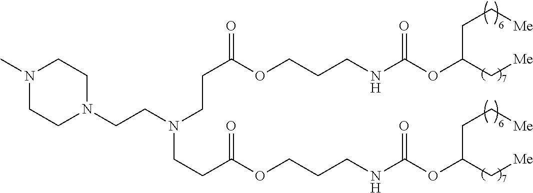

13-3-C8

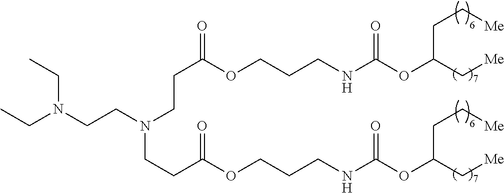

14-3-C8

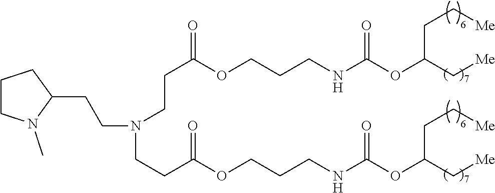

16-3-C8

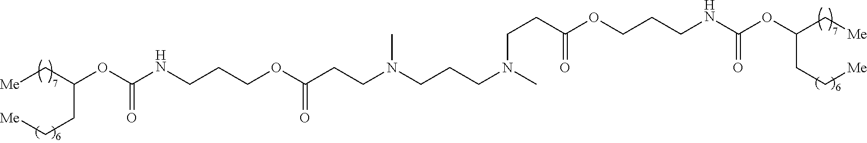

15-3-C8

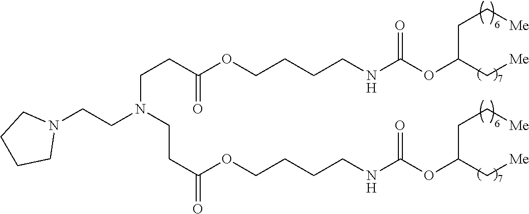

3-4-C8

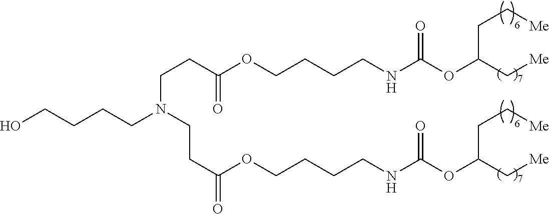

5-4-C8

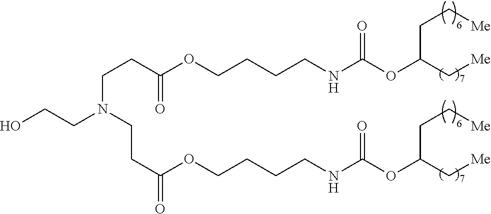

6-4-C8

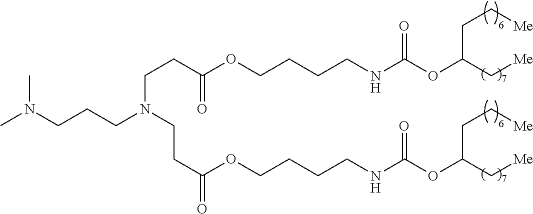

7-4-C8

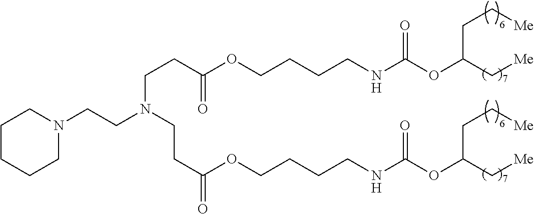

8-4-C8

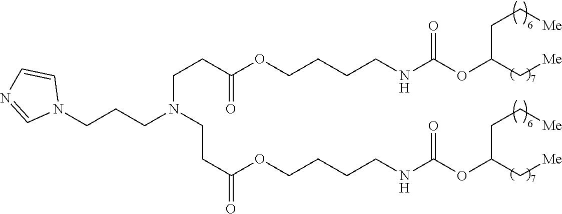

9-4-C8

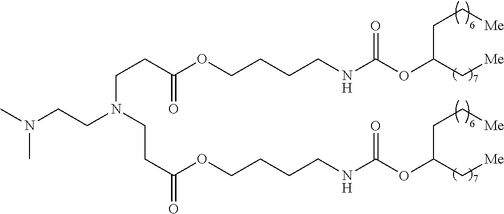

10-4-C8

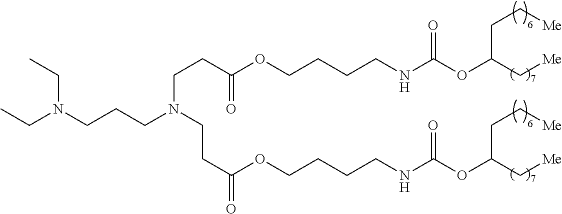

11-4-C8

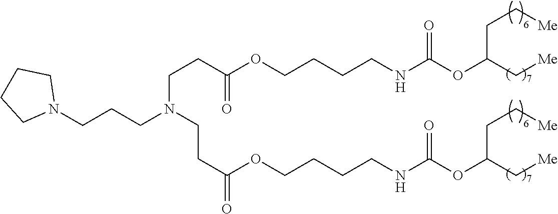

12-4-C8

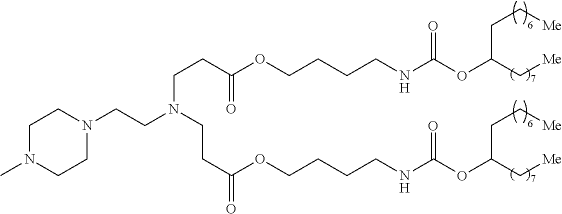

13-4-C8

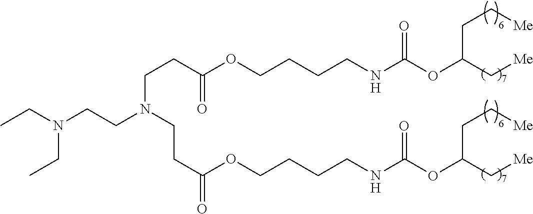

14-4-C8

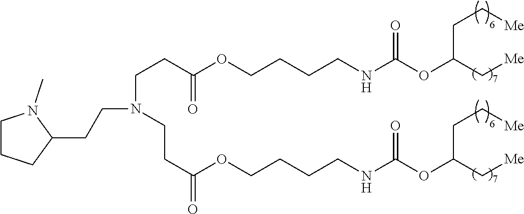

16-4-C8

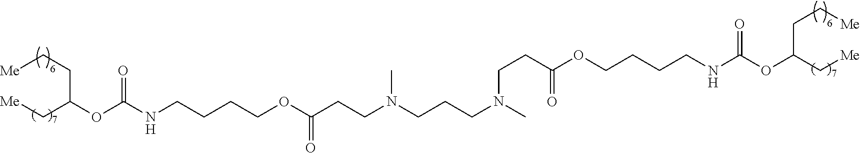

15-4-C8

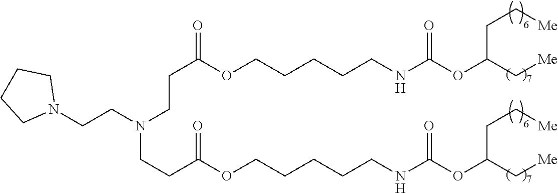

3-5-C8

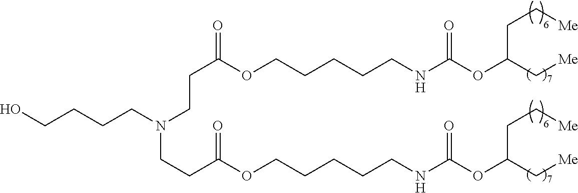

5-5-C8

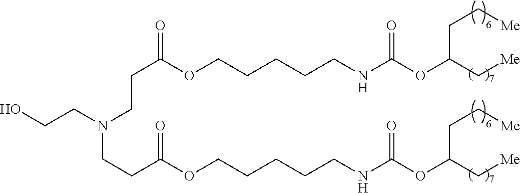

6-5-C8

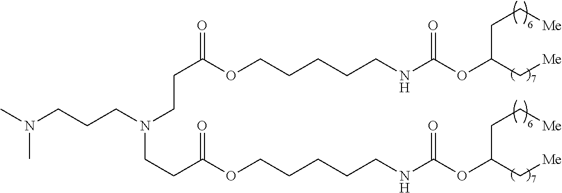

7-5-C8

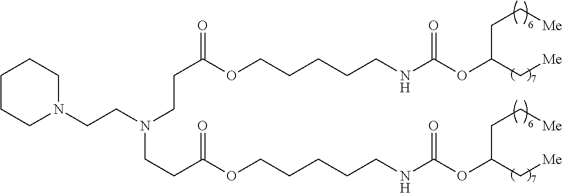

8-5-C8

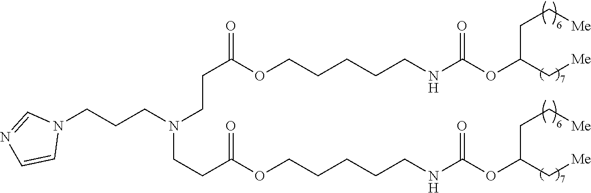

9-5-C8

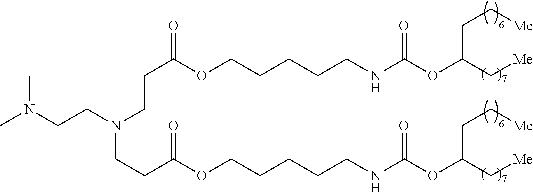

10-5-C8

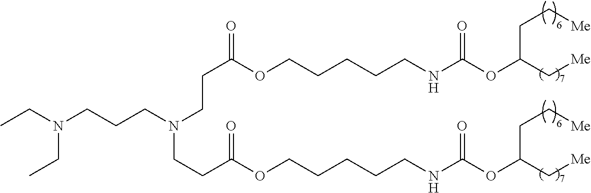

11-5-C8

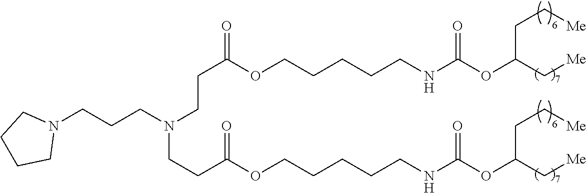

12-5-C8

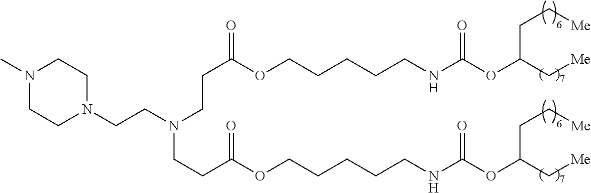

13-5-C8

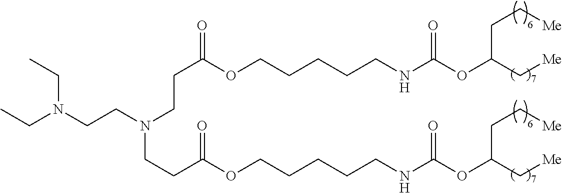

14-5-C8

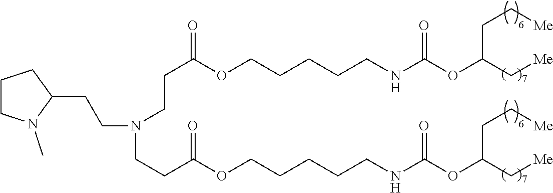

16-5-C8

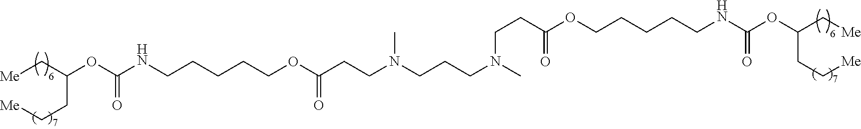

15-5-C8

### Performance Test

### 1. Lipid Compound Delivering Plasmid DNA Testing

The Lipid compounds were used to deliver plasmid DNA encoding green fluorescent protein (GFP) and firefly luciferase (Luc) in 293T cell lines. Lipid compounds 3-2-C8, 5-2-C8, 6-2-C8, 7-2-C8, 8-2-C8, 9-2-C8, 10-2-C8, 11-2-C8, 12-2-C8, 13-2-C8, 14-2-C8, 15-2-C8, 16-2-C8; 3-3-C8, 5-3-C8, 6-3-C8, 7-3-C8, 8-3-C8, 9-3-C8, 10-3-C8, 11-3-C8, 12-3-C8, 13-3-C8, 14-3-C8, 15-3-C8, 16-3-C8; 3-4-C8, 5-4-C8, 6-4-C8, 7-4-C8, 8-4-C8, 9-4-C8, 10-4-C8, 11-4-C8, 12-4-C8, 13-4-C8, 14-4-C8, 15-4-C8, 16-4-C8; 3-5-C8, 5-5-C8, 6-5-C8, 7-5-C8, 8-5-C8, 9-5-C8, 10-5-C8, 11-5-C8, 12-5-C8, 13-5-C8, 14-5-C8, 15-5-C8, 16-5-C8 were used as nucleic acid delivery materials to deliver pDNA-GFP-Luc.

The specific steps are as follows:

1) Cell culture: One day before the experiment, 293T cells were seeded in a 96-well cell culture plate (30%-40% density), when the cell density grew to about 70%, cell transfection was performed.

2) Preparation of LNP-pDNA-GFP-Luc lipid nanoparticles for cell transfection: Lipid compounds 3-2-C8, 5-2-C8, 6-2-C8, 7-2-C8, 8-2-C8, 9-2-C8, 10-2-C8, 11-2-C8, 12-2-C8, 13-2-C8, 14-2-C8, 15-2-C8, 16-2-C8; 3-3-C8, 5-3-C8, 6-3-C8, 7-3-C8, 8-3-C8, 9-3-C8, 10-3-C8, 11-3-C8, 12-3-C8, 13-3-C8, 14-3-C8, 15-3-C8, 16-3-C8; 3-4-C8, 5-4-C8, 6-4-C8, 7-4-C8, 8-4-C8, 9-4-C8, 10-4-C8, 11-4-C8, 12-4-C8, 13-4-C8, 14-4-C8, 15-4-C8, 16-4-C8; 3-5-C8, 5-5-C8, 6-5-C8, 7-5-C8, 8-5-C8, 9-5-C8, 10-5-C8, 11-5-C8, 12-5-C8, 13-5-C8, 14-5-C8, 15-5-C8, 16-5-C8 and dioleoyl phosphatidyl ethanolamine (DOPE), cholesterol, distearoylphosphatidylacetamide-polyethylene glycol (DSPE-PEG) were dissolved in anhydrous ethanol at a certain concentration, the components were uniformly mixed according to a certain molar ratio of lipid compound:Cholesterol:DOPE:DSPE-PEG=40:48:10:2, at the same time, a proper amount of pDNA-GFP-Luc was dissolved in sodium acetate buffer (the volume of sodium acetate buffer was twice the total volume of the lipid mixture, pH=5.3). The pDNA-GFP-Luc dissolved in the buffer was absorbed into the lipid mixture solution, and quickly mixed evenly to assemble into lipid nanoparticles, the mixed solution was incubated at room temperature for 15 min. Then, the volume was diluted 2-3 times with sterile PBS and added to the cell culture medium for transfection (LNP containing 200 ng of pDNA-GFP-Luc was transfected into each well). The nitrogen-phosphorus ratio (N/P ratio) of the ionizable lipid to pDNA was 24:1, which was the optimal ratio, that is, the molar ratio between the protonated amino group on the ionized lipid and the phosphate group on the DNA. Negative control group: 293T cells were cultured normally without transfection.

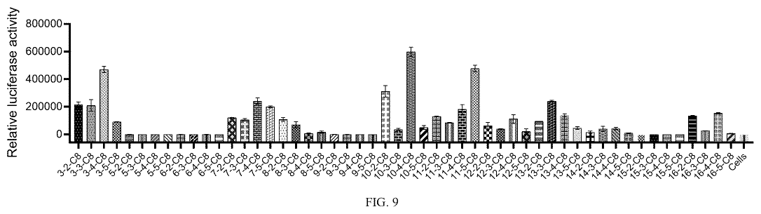

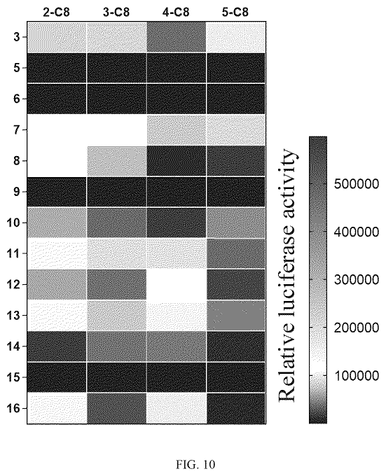

3) Cell transfection efficiency analysis: 48 h after transfection, the expression of the green fluorescent protein was detected by fluorescence microscopy; then, the 96-well cell culture plate was placed on ice to lyse the cells for 30 min, after centrifugation, the supernatant liquid was taken and the firefly luciferase substrate was added, the firefly luciferase activity (chemiluminescence) was detected by a microplate reader. FIG. 9 is the relative luciferase activity of the lipid compounds. FIG. 10 is the heat map of the relative luciferase activity of the lipid compounds. From FIG. 9 and FIG. 10, it can be seen that the expression of firefly luciferase in the negative control is the lowest, and most of the lipid compounds synthesized in this invention have strong transfection efficiency. Among them, 18 out of 52 ionizable lipids exhibit a luciferase expression level above 100,000 relative luciferase unit (RLU), accounting for 35% of the total lipids; 8 out of 52 ionizable lipids exhibit an expression level above 200,000 RLU, accounting for 15% of the total lipids; and 3 out of 52 ionizable lipids exhibits an expression level above 400,000 RLU, accounting for 6% of the total lipids. This demonstrates the reliability and high efficiency of the overall chemical structure design for these branched ionizable lipids.

### 2. The Test of the Particle Size, Polymer Dispersion Coefficient, and Zeta Potential of the Lipid Compound Nanoparticles.

Lipid compounds 3-2-C8, 3-3-C8, 3-4-C8, 3-5-C8, 10-2-C8, 10-3-C8, 10-4-C8 and 10-5-C8 were used as nanocarrier materials to encapsulate pDNA-GFP-Luc to test the particle size (Size), polymer dispersion index (PDI) and Zeta potential of the lipid nanoparticles.

1) The experimental steps are as follows:

Ionizable lipids 3-2-C8, 3-3-C8, 3-4-C8, 3-5-C8, 10-2-C8, 10-3-C8, 10-4-C8, 10-5-C8 and dioleoyl phosphatidyl ethanolamine (DOPE), cholesterol, distearoylphosphatidylacetamide-polyethylene glycol (DSPE-PEG) were dissolved in anhydrous ethanol at a certain concentration, the components were uniformly mixed according to a certain molar ratio of lipid compound:Cholesterol:DOPE:DSPE-PEG=40:48:10:2, at the same time, a proper amount of pDNA-GFP-Luc was dissolved in sodium acetate buffer (the volume of sodium acetate buffer was twice the total volume of lipid mixture, pH=5.3), and the pDNA-GFP-Luc dissolved in the buffer was taken into the lipid mixture solution, and quickly mixed to assemble into lipid nanoparticles, the mixed solution was incubated at room temperature for 15 min, and the particle size and potential were tested by dynamic light scattering instrument (DLS). The nitrogen-phosphorus ratio (N/P ratio) of ionizable lipid to pDNA (24:1) was the optimal ratio, that is, the molar ratio between the protonated amino group on the ionized lipid and the phosphate group on the DNA. 2) Analysis of experimental results:

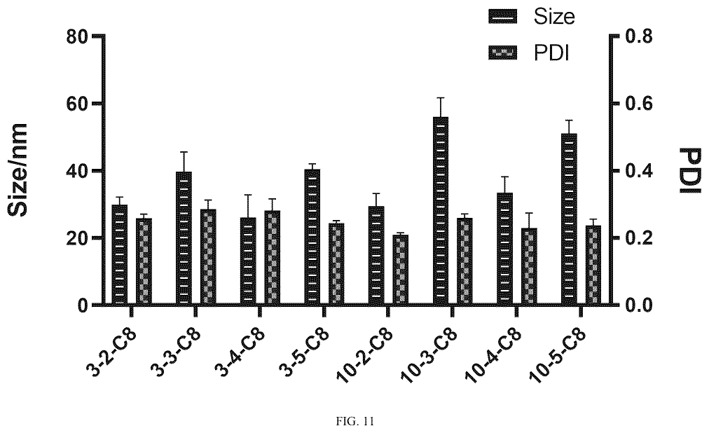

FIG. 11 is the particle size test diagram of nanoparticles prepared by lipid compounds. Table 2 is the Zeta potential test results of nanoparticles prepared by lipid compounds.

TABLE 2
Zeta potential test results of lipid compounds

| Main lipid | Zeta potential (mV) |
|---|---|
| 3-2-C8 | 13.0 ± 1.56 |
| 3-3-C8 | 23.1 ± 1.37 |
| 3-4-C8 | 20.2 ± 3.07 |
| 3-5-C8 | 17.4 ± 3.35 |
| 10-2-C8 | 20.7 ± 2.77 |
| 10-3-C8 | 19.4 ± 1.56 |
| 10-4-C8 | 23.5 ± 4.87 |
| 10-5-C8 | 28.7 ± 4.36 |

It can be seen from FIG. 11 and Table 2 that the test results show that the particle size of the selected eight ionizable lipids is between 30 nm-60 nm, the PDI is between 0.1-0.3, and the Zeta potential is positive at pH=5.3. The charge is 13.0±1.56-28.7±4.36 mV, which is consistent with the characteristics of lipid nanoparticles, the smaller particle sizes can exert the high permeability and long retention effect (EPR), leading to better accumulation in the tumor sites.

### 3. Lipid Compound Delivery RNA Test

Lipid compounds were used to deliver self-amplifying RNA (Rep RNA-GFP-Luc) encoding green fluorescent protein (GFP) and firefly luciferase (Luc) in the 293 T cell line. Lipids 3-2-C8, 5-2-C8, 6-2-C8, 7-2-C8, 8-2-C8, 9-2-C8, 10-2-C8, 11-2-C8, 12-2-C8, 13-2-C8, 14-2-C8, 15-2-C8, 16-2-C8; 3-3-C8, 5-3-C8, 6-3-C8, 7-3-C8, 8-3-C8, 9-3-C8, 10-3-C8, 11-3-C8, 12-3-C8, 13-3-C8, 14-3-C8, 15-3-C8, 16-3-C8; 3-4-C8, 5-4-C8, 6-4-C8, 7-4-C8, 8-4-C8, 9-4-C8, 10-4-C8, 11-4-C8, 12-4-C8, 13-4-C8, 14-4-C8, 15-4-C8, 16-4-C8; 3-5-C8, 5-5-C8, 6-5-C8, 7-5-C8, 8-5-C8, 9-5-C8, 10-5-C8, 11-5-C8, 12-5-C8, 13-5-C8, 14-5-C8, 15-5-C8, 16-5-C8 were used respectively as nucleic acid delivery materials to deliver Rep RNA-GFP-Luc.

The specific steps are as follows:

1) Cell culture: One day before the experiment, 293T cells were seeded in a 96-well cell culture plate (30%-40% density), when the cell density grew to about 70%, cell transfection was performed.

2) Preparation of LNP-repRNA-GFP-Luc lipid nanoparticles for cell transfection: Lipid compounds 3-2-C8, 5-2-C8, 6-2-C8, 7-2-C8, 8-2-C8, 9-2-C8, 10-2-C8, 11-2-C8, 12-2-C8, 13-2-C8, 14-2-C8, 15-2-C8, 16-2-C8; 3-3-C8, 5-3-C8, 6-3-C8, 7-3-C8, 8-3-C8, 9-3-C8, 10-3-C8, 11-3-C8, 12-3-C8, 13-3-C8, 14-3-C8, 15-3-C8, 16-3-C8; 3-4-C8, 5-4-C8, 6-4-C8, 7-4-C8, 8-4-C8, 9-4-C8, 10-4-C8, 11-4-C8, 12-4-C8, 13-4-C8, 14-4-C8, 15-4-C8, 16-4-C8; 3-5-C8, 5-5-C8, 6-5-C8, 7-5-C8, 8-5-C8, 9-5-C8, 10-5-C8, 11-5-C8, 12-5-C8, 13-5-C8, 14-5-C8, 15-5-C8, 16-5-C8 and dioleoyl phosphatidyl ethanolamine (DOPE), cholesterol, distearoylphosphatidylacetamide-polyethylene glycol (DSPE-PEG) were dissolved in anhydrous and non-enzymatic ethanol at a certain concentration, the components were uniformly mixed according to a certain molar ratio of lipid compound:Cholesterol:DOPE:DSPE-PEG=40:48:10:2, at the same time, a proper amount of repRNA-GFP-Luc was dissolved in sodium acetate buffer (the volume of sodium acetate buffer was twice the total volume of the lipid mixture, pH=5.3). The repRNA-GFP-Luc dissolved in the buffer was added into the lipid mixture solution, and quickly mixed evenly to assemble into lipid nanoparticles, the mixed solution was incubated at room temperature for 15 min. Then, the volume was diluted 2-3 times with sterile PBS and added to the cell culture medium for transfection (LNP containing 150 ng of repRNA-GFP-Luc was transfected into each well). The nitrogen-phosphorus ratio (N/P ratio) of the ionizable lipid to mRNA was 24:1, which was the optimal ratio, that is, the molar ratio between the protonated amino group on the ionized lipid and the phosphate group on the mRNA.

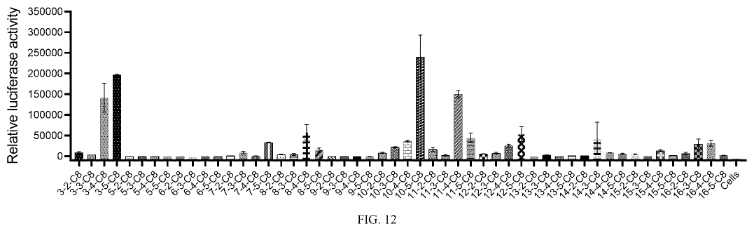

3) Cell transfection efficiency analysis: After 36 h of transfection, the expression of the green fluorescent protein was detected by fluorescence microscopy; then, the 96-well cell culture plate was placed on ice to lyse the cells for 30 min, after centrifugation, the supernatant liquid was taken and the firefly luciferase substrate was added, the firefly luciferase activity (chemiluminescence) was detected by a microplate reader. FIG. 12 is the relative luciferase activity after transfection with nanoparticles prepared by the lipid compounds. It can be seen from FIG. 12 that the negative control has the lowest expression of firefly luciferase, generally speaking, self-replicating mRNA contains more molecular sequences than conventional mRNA, and it has a larger molecular weight, so the expression time is longer, at the same time, lipid nanoparticles are also more difficult to load and express effectively, the ionizable lipids synthesized by the invention also have strong transfection efficiency for repRNA. Without optimization, the expression intensity of 4 out of 52 ionizable lipids exceeds 150,000 RLU, accounting for 8% of the total lipids. It shows that the ionizable lipids synthesized by the invention can effectively deliver and efficiently transfect DNA and RNA.

### 4. Ratio Optimization Test of Nanoparticle Component for RNA Delivery by Lipid Compounds

Ionizable lipid compounds 3-5-C8 and 10-5-C8 were used to deliver self-amplifying RNA encoding green fluorescent protein (GFP) and firefly luciferase (Luc) in the 293 T cell line, and a ratio optimization was performed for the component of nanoparticles.

1) The specific preparation steps are the same as the lipid compound delivery RNA test steps of performance test 3. The difference is that in this performance test, the experimental group is: ionizable lipid compound 3-5-C8 or 10-5-C8, dioleoyl phosphatidylethanolamine (DOPE), cholesterol, and distearoyl phosphatidylacetamide-polyethylene glycol (DSPE-PEG) are dissolved in anhydrous and non-enzymatic ethanol at a certain concentration, and mixed in four different molar ratios. Ratio A is ionizable lipid compound 3-5-C8 or 10-5-C8:Cholesterol:DOPE:DSPE-PEG=40:48:10:2; Ratio B is ionizable lipid compound 3-5-C8 or 10-5-C8:Cholesterol:DOPE:DSPE-PEG=30:28.5:10:0.75; Ratio C is ionizable lipid compound 3-5-C8 or 10-5-C8:Cholesterol:DOPE:DSPE-PEG=50:38.5:10:1.5; Ratio D is 3-5-C8 or 10-5-C8:Cholesterol:DOPE:DSPE-PEG=35:46:16:2.5.

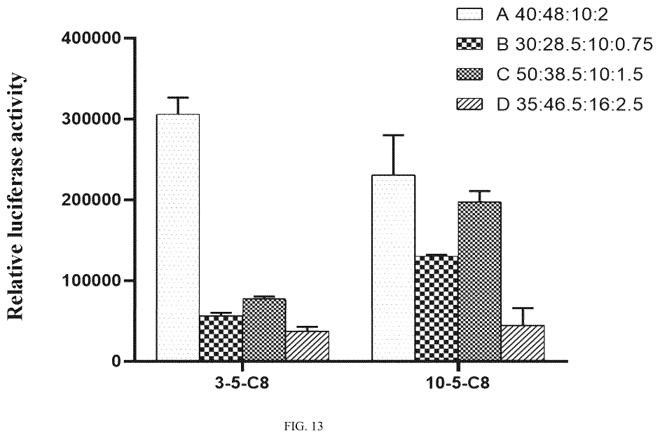

2) Cell transfection efficiency analysis: After 36 hours of transfection, the expression of the green fluorescent protein was detected by fluorescence microscopy; then, the 96-well cell culture plate was placed on ice to lyse the cells for 30 min, the supernatant liquid was taken after it was centrifuged, the firefly luciferase substrate was added, and the firefly luciferase activity (chemiluminescence) was detected by a microplate reader. FIG. 13 is the relative luciferase activity of the lipid compounds. It can be seen from FIG. 13 that the ratio A (ionizable lipid compound:Cholesterol:DOPE:DSPE-PEG=40:48:10:2) is the optimal ratio.

### 5. Optimization Test of the Neutral Phospholipids in the Nanoparticle for RNA Delivery by Lipid Compounds

Lipid compounds 3-5-C8 and 10-5-C8 were used to deliver self-amplifying RNA encoding green fluorescent protein (GFP) and firefly luciferase (Luc) in the 293T cell line to perform the optimization of the neutral phospholipids in nanoparticles.

1) The specific preparation steps are the same as the lipid compound delivery RNA test steps of performance test 3, the difference is that in this performance test, the experimental group is: lipid compound 3-5-C8 or 10-5-C8 and dioleoyl phosphatidyl ethanolamine (DOPE) or distearoylphosphatidylcholine (DSPC), cholesterol (Cholesterol), and distearoylphosphatidylacetamide-polyethylene glycol (DSPE-PEG) were dissolved in anhydrous and non-enzymatic ethanol according to a certain concentration, and the use ratio is ionizable lipid compound 3-5-C8 or 10-5-C8:Cholesterol:DOPE or DSPC or DOPC:DSPE-PEG=40:48:10:2.

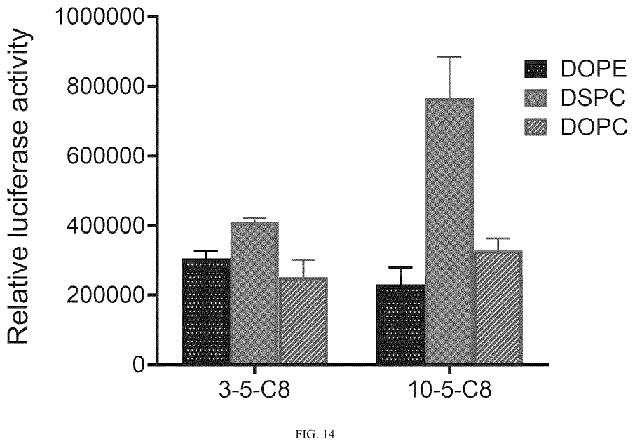

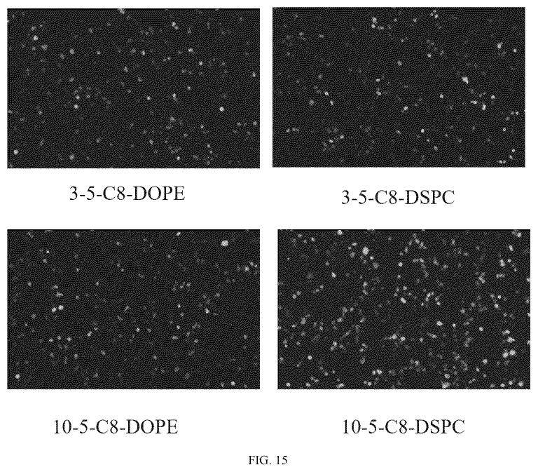

2) Cell transfection efficiency analysis: After 36 h of transfection, the expression of green fluorescent protein was detected by fluorescence microscopy; then, the 96-well cell culture plate was placed on ice to lyse the cells for 30 min. After centrifugation, the supernatant liquid was taken and the firefly luciferase substrate was added. The firefly luciferase activity (chemiluminescence) was detected by a microplate reader. FIG. 14 is the relative luciferase activity test results of the lipid compounds. FIG. 15 is the fluorescence microscope result of the lipid compounds. It can be seen from FIG. 15 that when DSPC was used as a helper phospholipid, the expression levels of 3-5-C8 and 10-5-C8 increased, and the enhancement of 10-5-C8 was the most obvious, its value reached 800,000 RLU.

6. Lipid Compound Delivering siRNA Test

Lipid compounds were used to deliver siRNA (siLuc) encoding firefly luciferase (Luc) in the B16F10-Luc cell line. Lipids 3-2-C8, 5-2-C8, 6-2-C8, 7-2-C8, 8-2-C8, 9-2-C8, 10-2-C8, 11-2-C8, 12-2-C8, 13-2-C8, 14-2-C8, 15-2-C8, 16-2-C8; 3-3-C8, 5-3-C8, 6-3-C8, 7-3-C8, 8-3-C8, 9-3-C8, 10-3-C8, 11-3-C8, 12-3-C8, 13-3-C8, 14-3-C8, 15-3-C8, 16-3-C8; 3-4-C8, 5-4-C8, 6-4-C8, 7-4-C8, 8-4-C8, 9-4-C8, 10-4-C8, 11-4-C8, 12-4-C8, 13-4-C8, 14-4-C8, 15-4-C8, 16-4-C8; 3-5-C8, 5-5-C8, 6-5-C8, 7-5-C8, 8-5-C8, 9-5-C8, 10-5-C8, 11-5-C8, 12-5-C8, 13-5-C8, 14-5-C8, 15-5-C8, 16-5-C8 were used respectively as nucleic acid delivery materials to deliver siLuc.

The specific steps are as follows:

1) Cell culture: One day before the experiment, B16F10-Luc cells were seeded in a 96-well cell culture plate (30%-40% density), when the cell density grew to about 70%, cell transfection was performed.

2) Preparation of LNP-siLuc lipid nanoparticles for cell transfection to silence the expression of Luc in cell lines: Ionizable lipids 3-2-C8, 5-2-C8, 6-2-C8, 7-2-C8, 8-2-C8, 9-2-C8, 10-2-C8, 11-2-C8, 12-2-C8, 13-2-C8, 14-2-C8, 15-2-C8, 16-2-C8; 3-3-C8, 5-3-C8, 6-3-C8, 7-3-C8, 8-3-C8, 9-3-C8, 10-3-C8, 11-3-C8, 12-3-C8, 13-3-C8, 14-3-C8, 15-3-C8, 16-3-C8; 3-4-C8, 5-4-C8, 6-4-C8, 7-4-C8, 8-4-C8, 9-4-C8, 10-4-C8, 11-4-C8, 12-4-C8, 13-4-C8, 14-4-C8, 15-4-C8, 16-4-C8; 3-5-C8, 5-5-C8, 6-5-C8, 7-5-C8, 8-5-C8, 9-5-C8, 10-5-C8, 11-5-C8, 12-5-C8, 13-5-C8, 14-5-C8, 1:15-5-C8, 16-5-C8 and distearoylphosphatidylcholine (DSPC), cholesterol, distearoylphosphatidylacetamide-polyethylene glycol (DSPE-PEG) were dissolved in anhydrous and non-enzymatic ethanol at a certain concentration. The components were uniformly mixed according to a certain molar ratio of lipid compound:Cholesterol:DSPC:DSPE-PEG=40:48:10:2, at the same time, a proper amount of siLuc was dissolved in sodium acetate buffer (the volume of sodium acetate buffer was twice the total volume of the lipid mixture, pH=5.3), the siLuc dissolved in the buffer was added into the lipid mixture solution, and quickly mixed evenly to assemble into lipid nanoparticles, the mixed solution was incubated at room temperature for 15 min. Then, the volume was diluted 2-3 times with sterile PBS and added to the cell culture medium for transfection (LNP containing 50 ng of siLuc was transfected into each well). The nitrogen-phosphorus ratio (N/P ratio) of the ionizable lipid to siRNA was 24:1, which was the optimal ratio, that is, the molar ratio between the protonated amino group on the ionized lipid and the phosphate group on the siRNA.

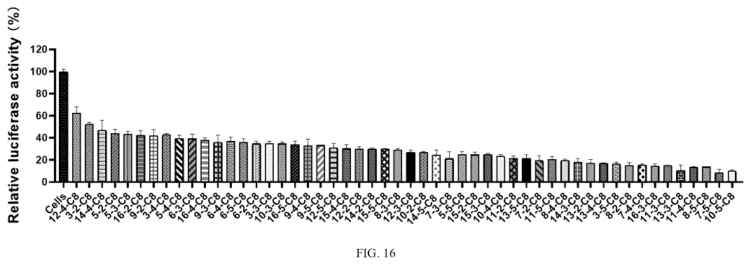

3) Analysis of cell transfection efficiency: After 24 h of transfection, the 96-well cell culture plate was placed on ice to lyse the cells for 30 minutes, after centrifugation, the supernatant was taken and the firefly luciferase substrate was added, the firefly luciferase activity (chemiluminescence) was detected by a microplate reader. FIG. 16 is the relative luciferase activity percentage of the lipid compounds. As shown in FIG. 16, the negative control (Cells) group expresses firefly luciferase at 100%, and all lipids synthesized by this invention are effective for siRNA silencing, with silencing efficiency concentrated between 70-90%. There are 3-5 lipids with efficiency above 90%, indicating that the ionizable lipids synthesized in this invention can effectively deliver siRNA and show good effects in delivering various RNAs and DNAs.

### 7. Lipid Compound In Vivo Delivery Test in Mice

Oligo-DNA with Cyanine 5 (Cy5) fluorescence was delivered in vivo in C57BL/6 mice using the ionizable lipid compounds 3-4-C8, 8-4-C8, 10-4-C8, 11-4-C8, and injected into the tail vein of mice. After 6-24 hours of blood circulation, the mice were sacrificed and the organs were lysed to detect the distribution of nanoparticles in various organs.

The specific steps are as follows:

1) Preparation of LNP-Cy5-Oligo lipid nanoparticles for tail vein injection in mice: The specific steps are the same with the lipid compound delivering plasmid DNA test in performance test 1, the difference is that in this performance test, the experimental group is: ionizable lipid compounds 3-4-C8, 8-4-C8, 10-4-C8 or 11-4-C8 and dioleoyl phosphatidylethanolamine (DOPE), cholesterol, distearoyl phosphatidylacetamide-polyethylene glycol (DSPE-PEG) were dissolved in anhydrous ethanol according to a certain concentration, the use ratio of ionizable lipid compounds 3-4-C8, 8-4-C8, 10-4-C8 or 11-4-C8:Cholesterol:DOPE:DSPE-PEG=40:48:10:2. 5 μg Cy5-Oligo was dissolved in sodium acetate buffer (the volume of sodium acetate buffer was twice the total volume of lipid mixture, pH=5.3). Cy5-Oligo dissolved in buffer was added in lipid mixture solution, and quickly mixed to assemble into lipid nanoparticles, the mixed solution was incubated at room temperature for 15 min, and then dialyzed in ultrapure water for 0.5 h using a dialysis bag (14000 MW), finally, 10% glucose solution was added to the lipid nanoparticle solution to adjust the osmotic pressure for tail vein injection (n=3 mice in each group). Among them, the N/P ratio of ionizable lipid to mRNA (N/P ratio) was 24:1, which was the optimal ratio, that is, the molar ratio between the protonated amino group on the ionizable lipid and the phosphate group on the DNA.

2) Lipid nanoparticle distribution in Lysis of organs and fluorescence test: After 12 hours of tail vein injection, the mice were sacrificed, blood and heart, liver, spleen, lung, and kidney organs were taken, and the tissues were ground, 0.5% TriTon X-100 lysis solution was placed on ice for 30 min, after centrifugation, the supernatant was taken and the fluorescence was tested using a microplate reader.

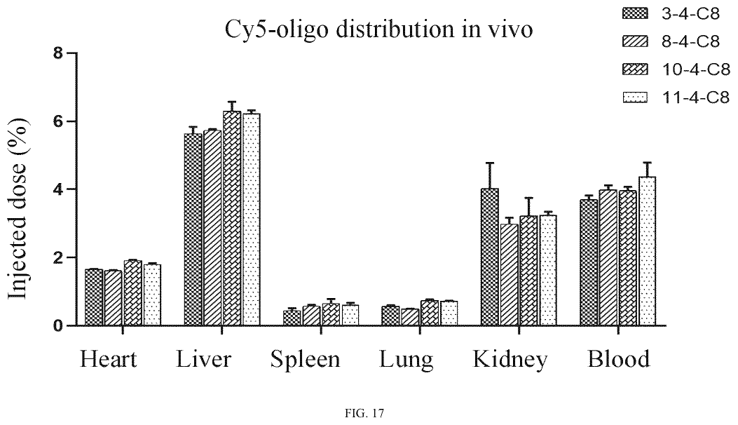

3) Analysis of experimental result: FIG. 17 is the distribution profile of lipid nanoparticles in various organs of mice. It can be seen that the nanoparticles will accumulate in the liver, and then in the kidney, which is consistent with the organ distribution characteristics of lipid nanoparticles, the nanoparticles were injected through the tail vein and distributed in various organs after blood circulation, indicating that the ionizable lipids synthesized by this invention can deliver nucleic acids in the body, laying the foundation for subsequent experiments.

### 8. RNA Imaging Test of Lipid Compound In Vivo Delivery in Mice

Ionizable lipid compounds 3-4-C8, 3-5-C8, 10-4-C8, 10-5-C8, and 11-4-C8 were used to deliver self-amplifying RNA (Rep RNA-Luc) encoding firefly luciferase (Luc) in C57BL/6 mice. After intramuscular injection in mice, the in vivo imaging system (IVIS) was used to detect on the 3rd, 5th, and 7th day after injection.

1) The specific steps are the same with the lipid compound delivering RNA test in performance test 3, the difference in this performance test, the experimental group: ionizable lipid compounds 3-4-C8, 3-5-C8, 10-4-C8, 10-5-C8 or 11-4-C8 and distearoyl phosphatidylcholine (DSPC), cholesterol (Cholesterol), distearoyl phosphatidylacetamide-polyethylene glycol (DSPE-PEG) or blank group are dissolved in anhydrous and non-enzymatic ethanol according to a certain concentration, the use of the ratio is ionizable lipid compounds 3-4-C8, 8-4-C8, 10-4-C8 or 11-4-C8:Cholesterol:DSPC:DSPE-PEG=40:48:10:2. A proper amount of repRNA-Luc was dissolved in sodium acetate buffer (the volume of sodium acetate buffer was twice the total volume of the lipid mixture, pH=5.3), the repRNA-Luc dissolved in the buffer was added into the lipid mixture solution and quickly mixed to assemble into lipid nanoparticles, the mixed solution was incubated at room temperature for 15 min, and then dialyzed in ultrapure water for 0.5 h using a dialysis bag (14000 MW), finally, 10% glucose solution was added to the lipid nanoparticle solution to adjust the osmotic pressure for intramuscular injection (LNP containing 150 ng of repRNA-Luc in each dose). Among them, the N/P ratio of ionizable lipid to mRNA (N/P ratio) is 24:1, which is the optimal ratio, that is, the molar ratio between the protonated amino group on the ionizable lipid and the phosphate group on the DNA.

2) Analysis of in vivo imaging results:

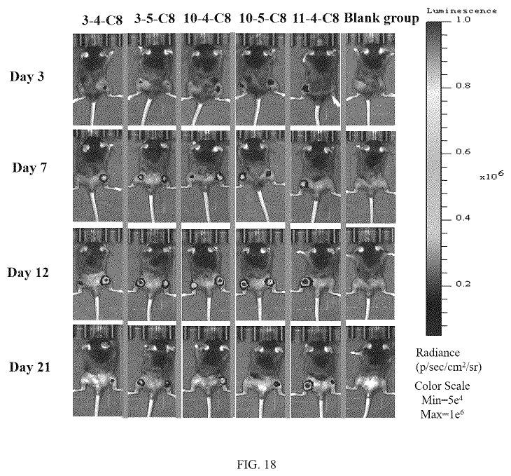

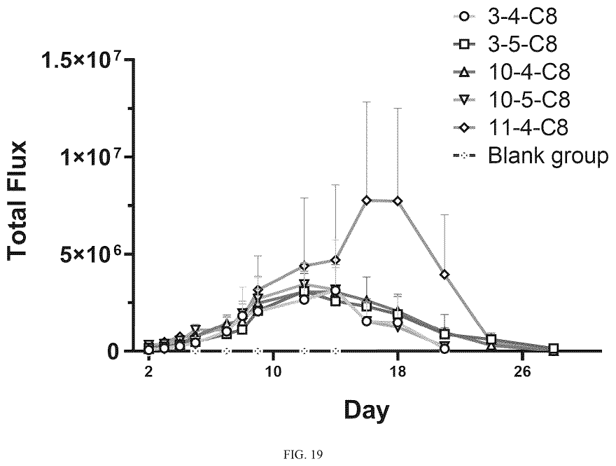

FIG. 18 is the detection map of the in vivo imaging system for lipid nanoparticles injected into mice. FIG. 19 is an injection time and total flux of the lipid compound. IVIS results showed that lipid nanoparticles 3-4-C8, 3-5-C8, 10-4-C8, 10-5-C8, or 11-4-C8 were successfully expressed on the 3rd day after intramuscular injection of repRNA-Luc, and the expression values increased with time, while the blank group had no expression. The expression value of repRNA-Luc reached a peak at 12-16 days after injection, while that of ordinary RNA-Luc reached a peak at 48 hours after injection according to the literature, indicating that the ionizable lipid in this application has a longer expression time in delivering this repRNA, the expression level is higher, which brings more lasting immune effects in the application of mRNA vaccine.

The invention provides a preparation method and application of a new branched tail lipid, which is an ionizable lipid, the tertiary amine or secondary amine head of this ionizable lipid can acquire a hydrogen proton under acidic conditions, becoming positive charged. It can bind with negatively charged RNA, DNA or small molecule drugs through electrostatic interactions, and then self-assemble with helper lipids into lipid nanoparticles (LNP) to deliver gene drugs to the target site. Based on a series of problems such as low efficiency and high toxicity encountered in the current gene drug delivery, the branched tail ionizable lipid ingeniously changed the hydrophobic tail of the ionizable lipid from a single alkyl chain to a double alkyl chain in its chemical structure design. Therefore: 1, the increase in the interval between lipids can enhance the protonation ability under the condition of endosomal pH; 2, increasing the cross-sectional area of the hydrophobic part of the lipid, the lipid is formed into a more conical structure, which can greatly increase the escape efficiency of the lipid nanoparticles in the intracellular endosome.

The invention provides a branched-chain ionizable lipid, and the prepared lipid nanoparticles can efficiently deliver mRNA, and pDNA, and efficiently transfect siRNA to specifically silence targeted gene expression in mammalian cells. When the LNP carrier reaches the intracellular environment through endocytosis, how to quickly escape in the endosome is a major problem to be solved in the efficient delivery system, the branched-chain ionizable lipid in the invention can increase the ionization degree of the lipid through the branch of the hydrophobic tail, improve the protonation ability, and expand the cross-sectional increase of the lipid tail, so that the nanoparticles are more conical in the structural assembly, thereby enhancing the endosome escape and improving the transfection efficiency. In addition, the ionizable lipid charge of the invention can change with the change of pH, and is electrically neutral under neutral conditions, reducing the cytotoxicity caused by too much positive charge, thereby increasing the stability of lipid nanoparticles, and avoiding rapid degradation in the body due to excessive positive charge, and help to extend the circulation time of the loaded nucleic acid drug and improve the pharmacokinetic characteristics.

The chemical structure of the branched-tail ionizable lipid can be roughly divided into a hydrophilic amino head, a central linker group, and a hydrophobic alkyl tail. Different from the harsh and complex synthetic routes of traditional cationic lipids, the branched-tail ionizable lipid provided by this invention features a simple structure design and a clear reaction mechanism. By using Michael addition reaction under solvent-free conditions, a large variety of structurally distinct ionizable lipids can be obtained, facilitating high-throughput screening.

The above examples are the better implementation methods of the invention, but the implementation method in the invention is not limited by the above embodiments, any other changes, modifications, substitutions, combinations, and simplifications that do not deviate from the spiritual essence and principle of the invention should be equivalent replacement methods and are included in the protection range of the invention.

What is claimed is:

1. A lipid compound, wherein a structure of the lipid compound is shown in a Formula (I);
A-(B)ₙ (I);
in the Formula (I), A is selected from a structure shown in a Formula (1)-a Formula (18), B is selected from a structure shown in a Formula (19), and n is selected from a positive integer of 2-4; 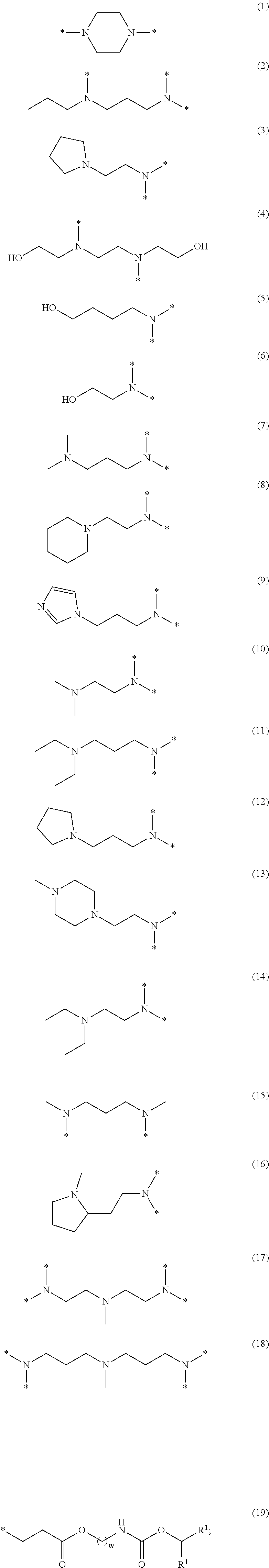
and
in the Formula (19), R¹ is selected from an alkyl or an alkenyl group of C6-C12, and m is selected from a positive integer of 1-6.

2. The lipid compound according to claim 1, wherein in the Formula (I), A is selected from a structure shown in the Formula (3), the Formula (5)-the Formula (16), B is selected from the structure shown in the Formula (19), and n is 2; and
in the Formula (19), R¹ is selected from an alkyl group of C7-C9, and m is selected from a positive integer of 2-5.

3. The lipid compound according to claim 2, wherein the lipid compound comprises a compound with the following structures:

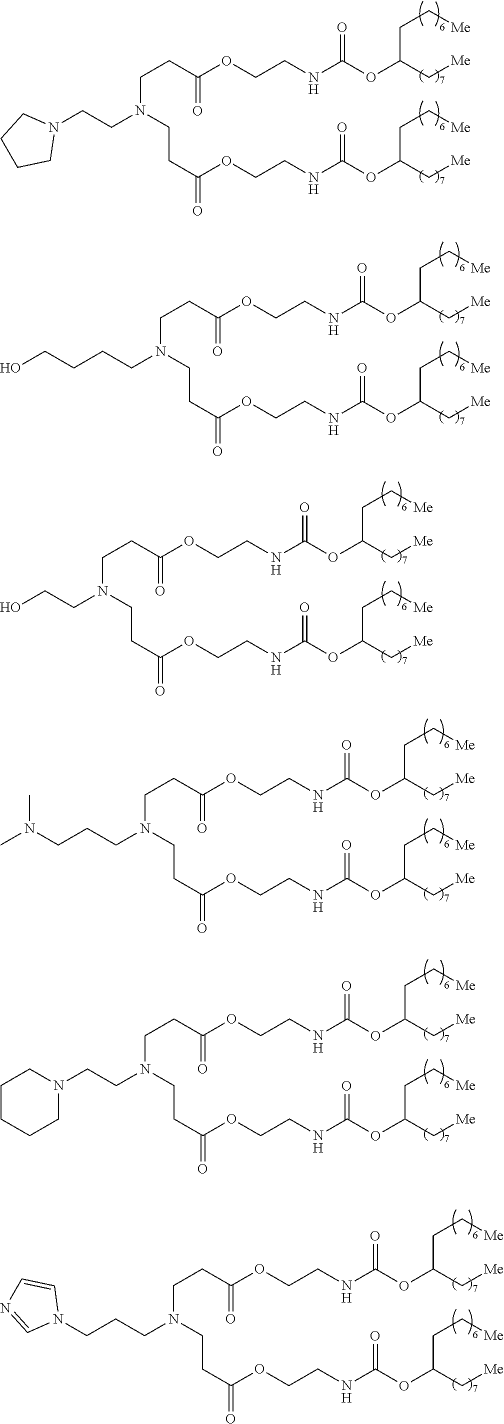

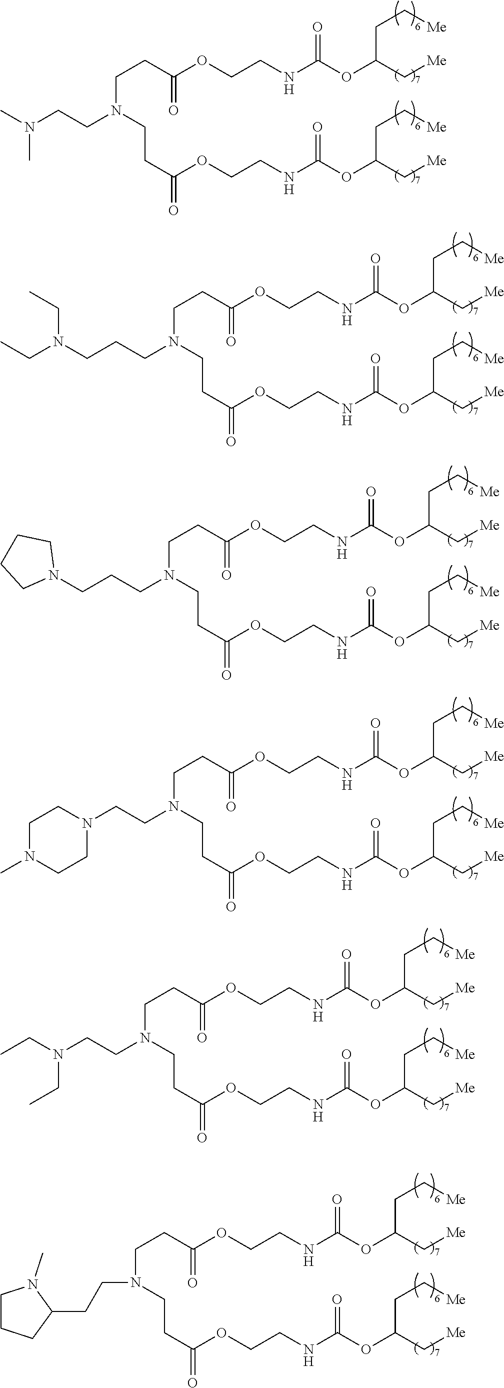

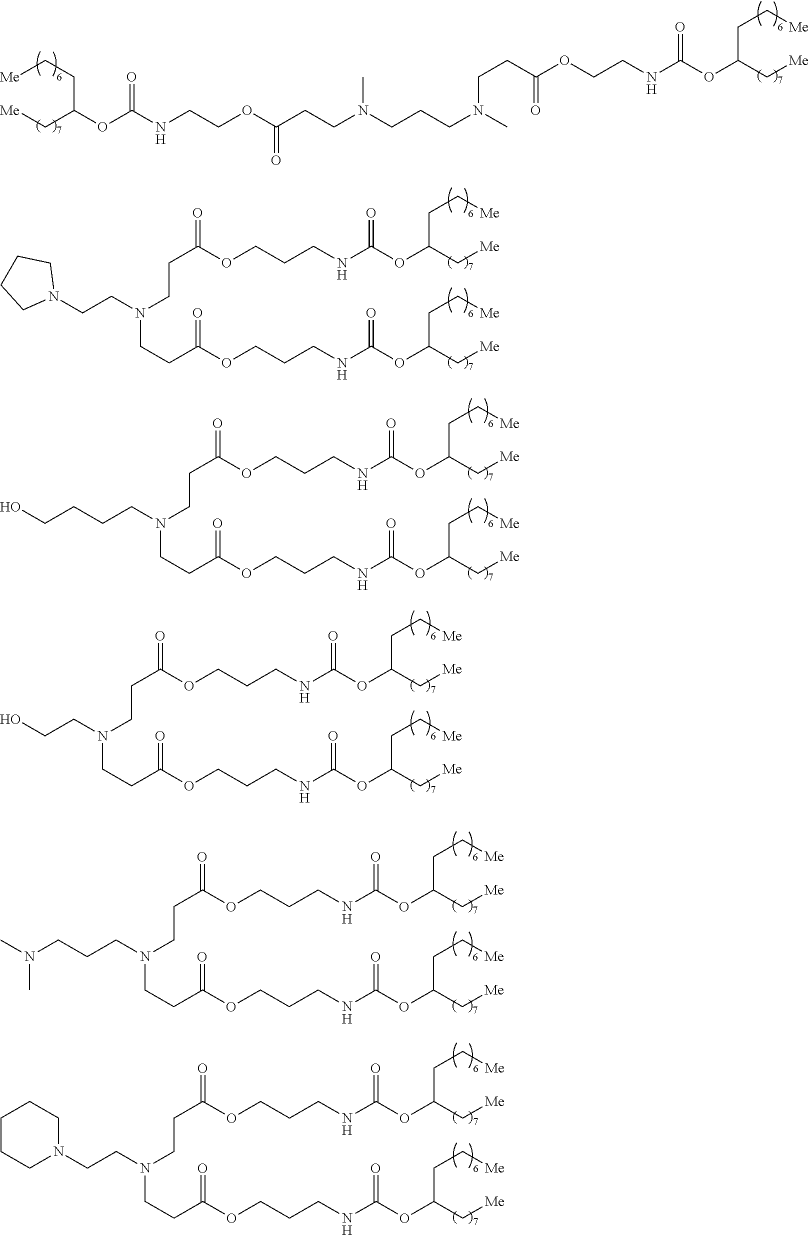

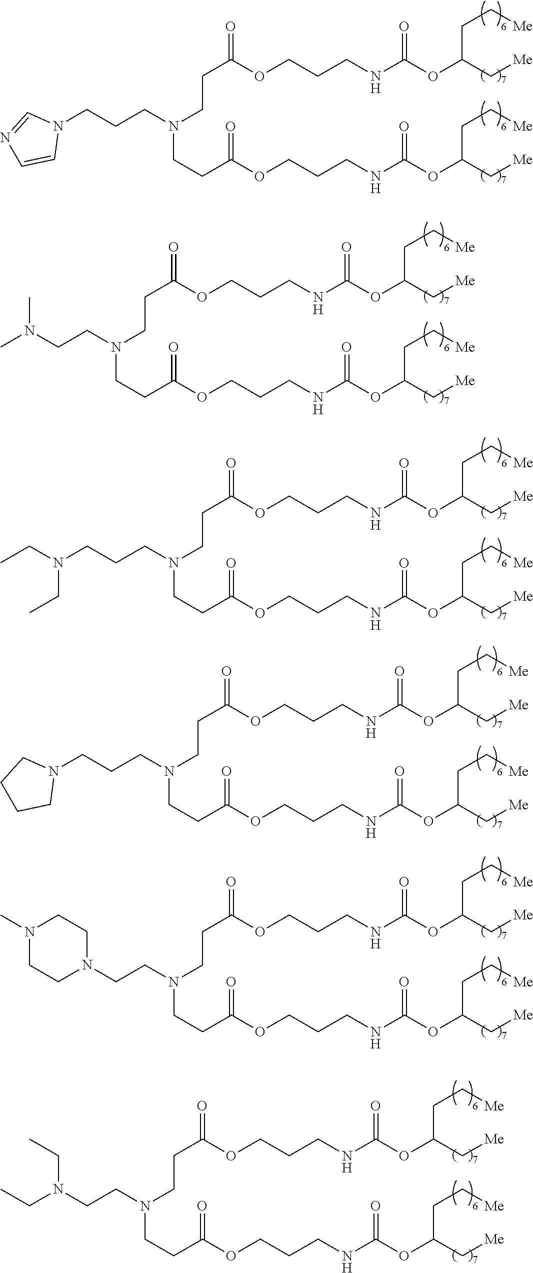

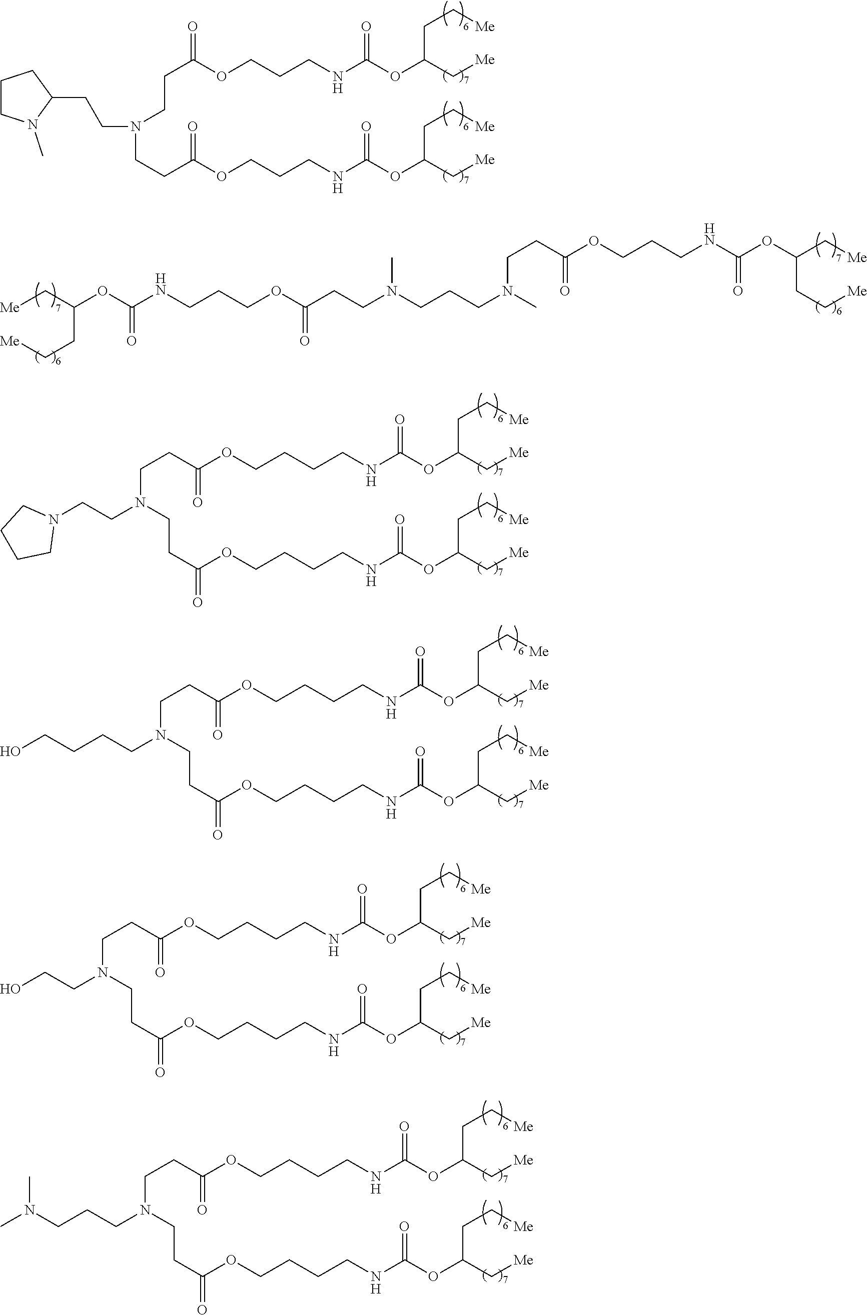

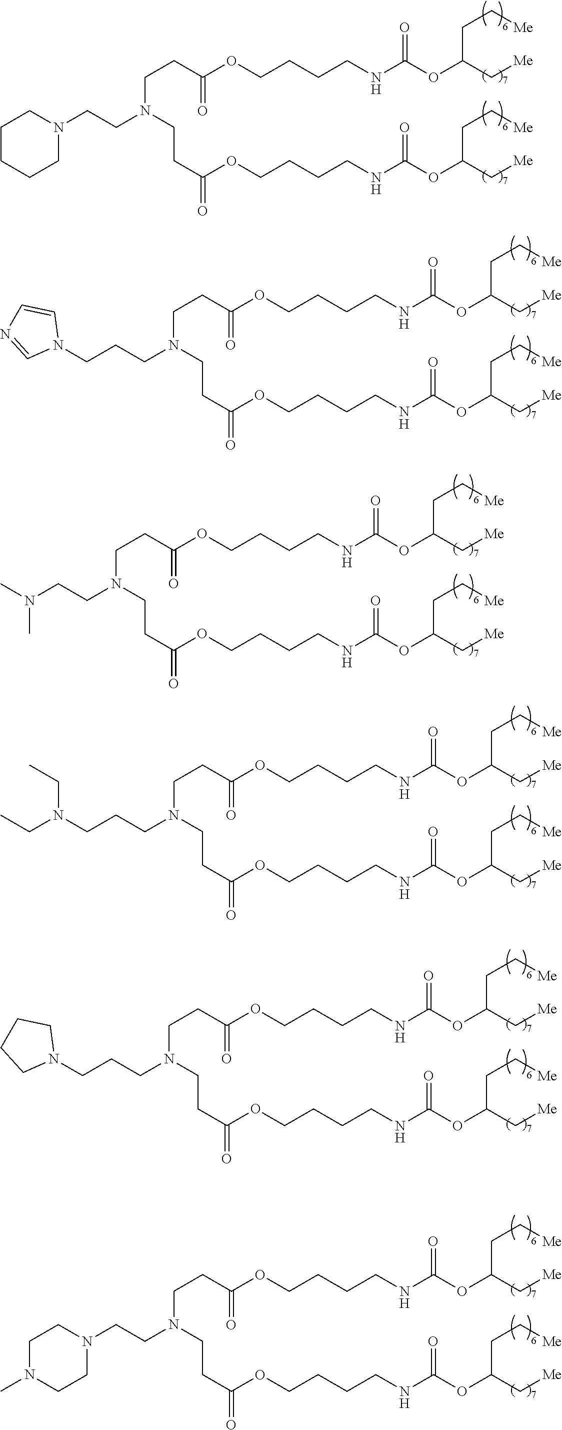

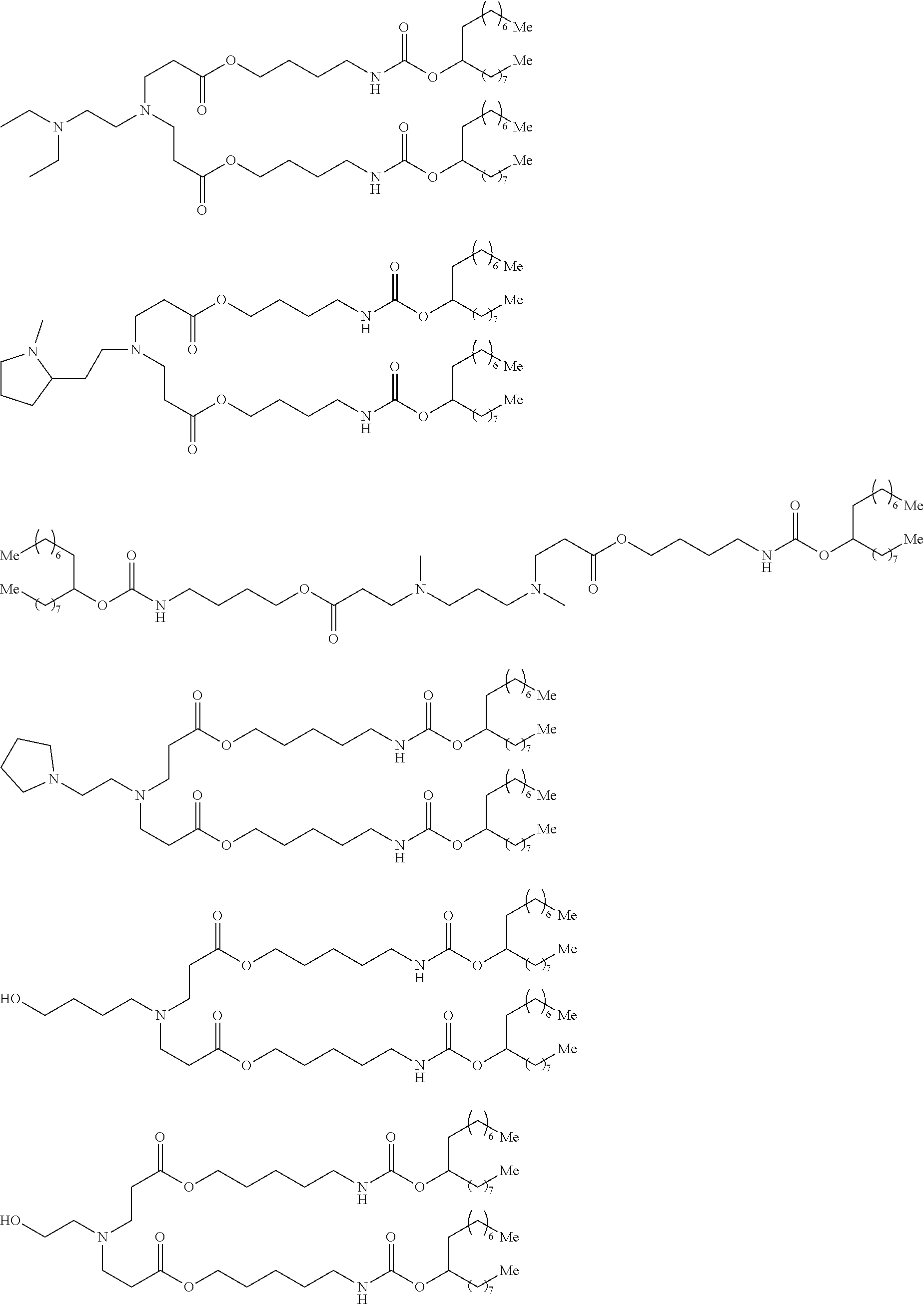

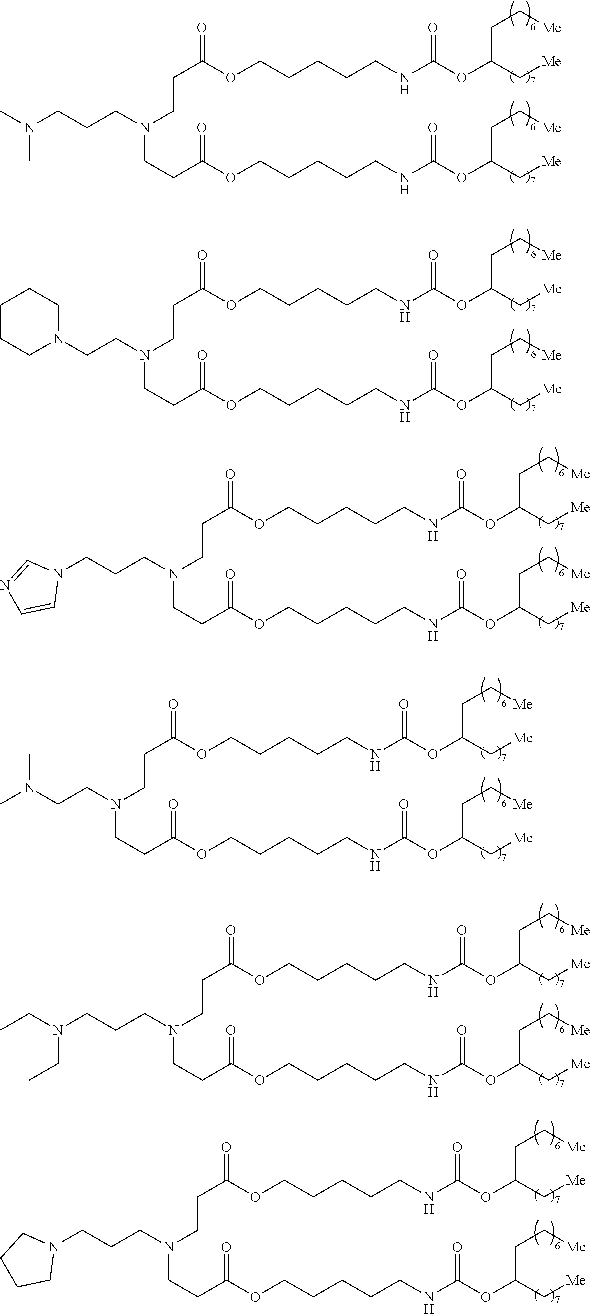

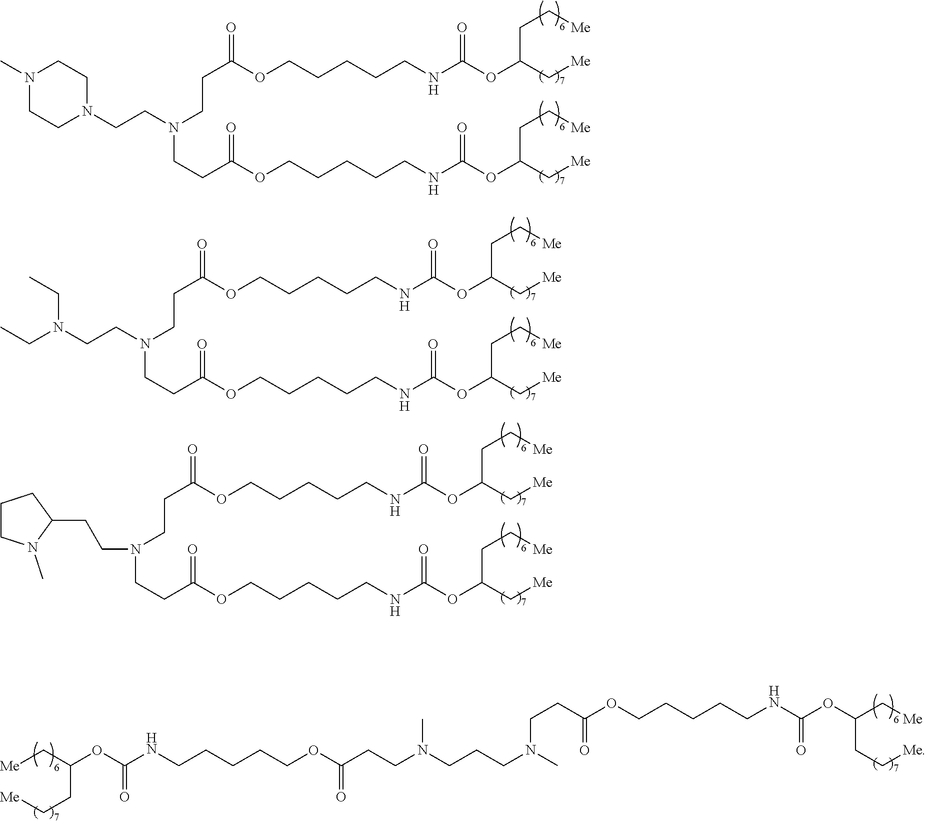

4. A preparation method for the lipid compound according to claim 1, comprising the following steps:
mixing a compound shown in a Formula (II) with a compound shown in a Formula (20)-a Formula (37), reacting, and obtaining the lipid compound; 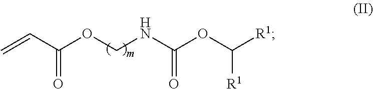
wherein in the Formula (II), R¹ is selected from the alkyl or the alkenyl group of C6-C12, m is selected from the positive integer of 1-6;

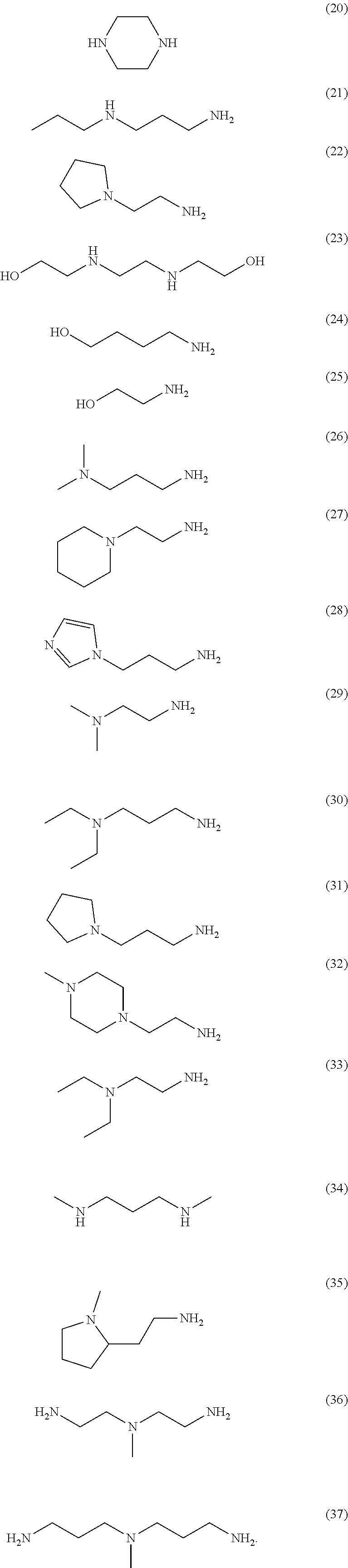

5. The preparation method for the lipid compound according to claim 4, wherein a molar ratio of an active hydrogen atom in the compound shown in the Formula (II) to the compound selected from the Formula (20)-the Formula (37) is (1-2):1.

6. A method of preparing a nucleic acid drug, a gene vaccine, a polypeptide or protein drug, and a small molecule drug, comprising using the lipid compound according to claim 1.

7. A method of preparing a drug carrier, comprising using the lipid compound according to claim 1.

8. The method according to claim 7, wherein drugs in the drug carrier comprise an RNA drug, a DNA drug, a polypeptide or protein drug, and a small molecule drug.

9. A drug composition, wherein the drug composition comprises the lipid compound according to claim 1, or a pharmaceutically acceptable salt of the lipid compound, or a stereoisomer of the lipid compound.

10. The drug composition according to claim 9, wherein the drug composition further comprises at least one of cholesterol and a cholesterol derivative, a helper phospholipid, and a polyethylene glycol (PEG) modified lipid.

11. The drug composition according to claim 10, wherein the helper phospholipid comprises at least one of egg yolk lecithin, hydrogenated egg yolk lecithin, soybean lecithin, hydrogenated soybean lecithin, sphingomyelin, phosphatidylethanolamine, dimyristoylphosphatidylcholine, dimyristoylphosphatidylglycerol, dipalmitoylphosphatidylcholine, distearoylphosphatidylcholine (DSPC), dioleoyl phosphatidyl ethanolamine (DOPE), dioleoylphosphatidylcholine (DOPC), dioleoylphosphatidylcholine, and dilauroylphosphatidylcholine.

12. The drug composition according to claim 10, wherein the PEG modified lipid comprises at least one of PEG modified phosphatidylethanolamine, PEG modified phosphatidic acid, PEG modified ceramide, PEG modified dialkylamine, PEG modified diacylglycerol, PEG modified dialkylglycerol, ALC-0159, and PEG modified products of the PEG modified phosphatidylethanolamine, the PEG modified phosphatidic acid, the PEG modified ceramide, the PEG modified dialkylamine, the PEG modified diacylglycerol, the PEG modified dialkylglycerol, and the ALC-0159.

13. The drug composition according to claim 10, wherein a molar ratio of the lipid compound, or the pharmaceutically acceptable salt of the lipid compound, or the stereoisomer of the lipid compound:the cholesterol and the cholesterol derivative:the helper phospholipid:the PEG modified lipid is (30-50):(30-50):(5-20):(1-2.5).

14. The drug composition according to claim 9, wherein the drug composition further comprises an active pharmaceutical ingredient; the active pharmaceutical ingredient comprises nucleic acid molecules and protein drugs, the nucleic acid molecules comprise a small interfering RNA (siRNA), a microRNA (miRNA), a messenger RNA (mRNA), a chemically modified mRNA (modRNA), a circular RNA (circRNA), an antisense RNA, a clustered regularly interspaced short palindromic repeats (CRISPR) guide RNA, a self-replicating RNA (repRNA), a cyclic dinucleotide (CDN), a polyIC, a CpG oligodeoxynucleotide (ODN), a plasmid DNA (pDNA), and a microcircular DNA; and the protein drugs comprise a cell colony stimulating factor, an interleukin, a lymphotoxin, an interferon protein, a tumor necrosis factor, an antibody, and a protein antigen.

15. The drug composition according to claim 14, wherein when the active pharmaceutical ingredient comprises the nucleic acid molecules, a ratio of nitrogen in the lipid compound, or the pharmaceutically acceptable salt of the lipid compound, or the stereoisomer of the lipid compound to phosphorus in the nucleic acid molecules is (4-32):1.

16. A method of preparing a nucleic acid drug, a gene vaccine, a polypeptide or protein drug, and a small molecule drug, comprising using the drug composition according to claim 9.

17. The preparation method for the lipid compound according to claim 4, wherein in the Formula (I) of the lipid compound, A is selected from a structure shown in the Formula (3), the Formula (5)-the Formula (16), B is selected from the structure shown in the Formula (19), and n is 2; and
in the Formula (19), R¹ is selected from an alkyl group of C7-C9, and m is selected from a positive integer of 2-5.

18. The preparation method for the lipid compound according to claim 17, wherein the lipid compound comprises a compound with the following structures:

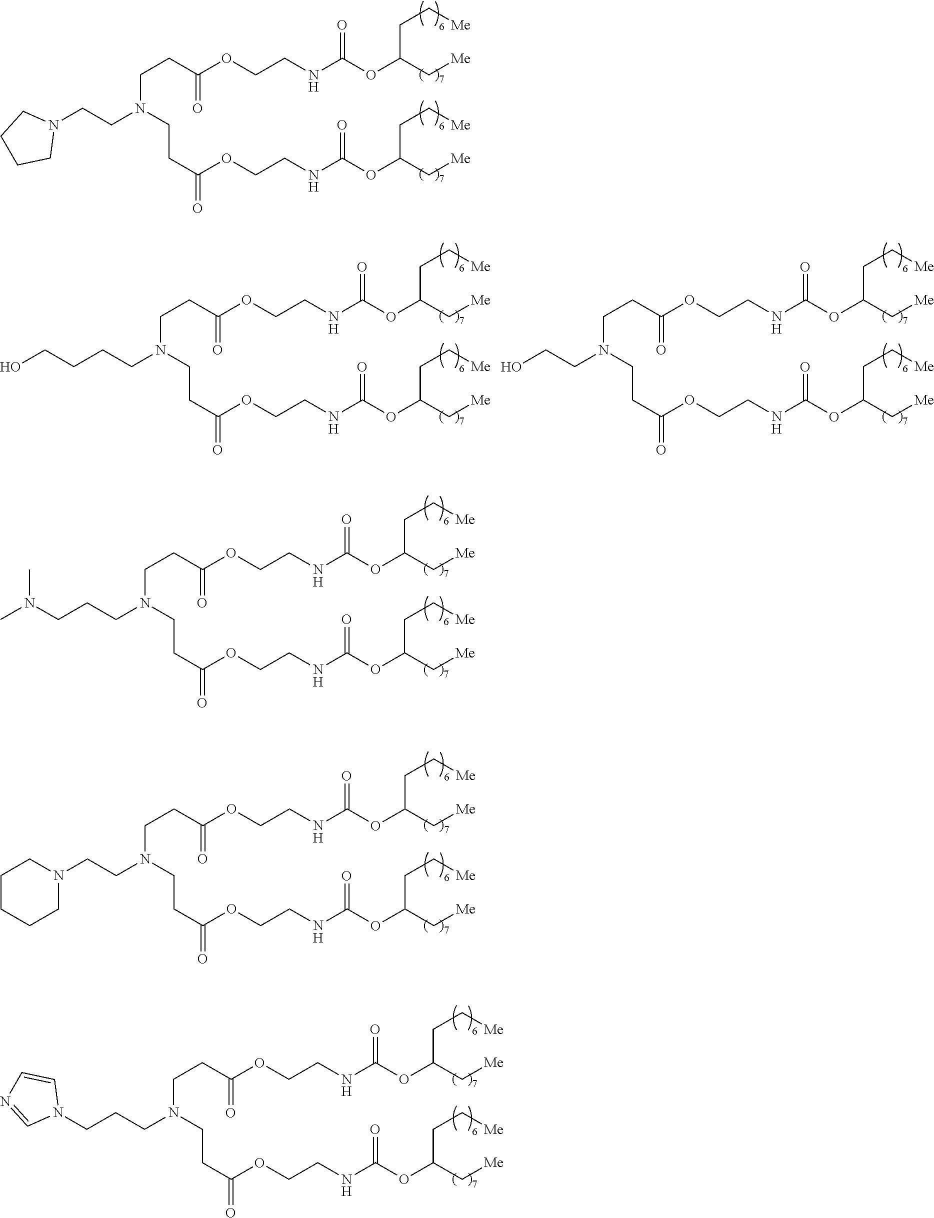

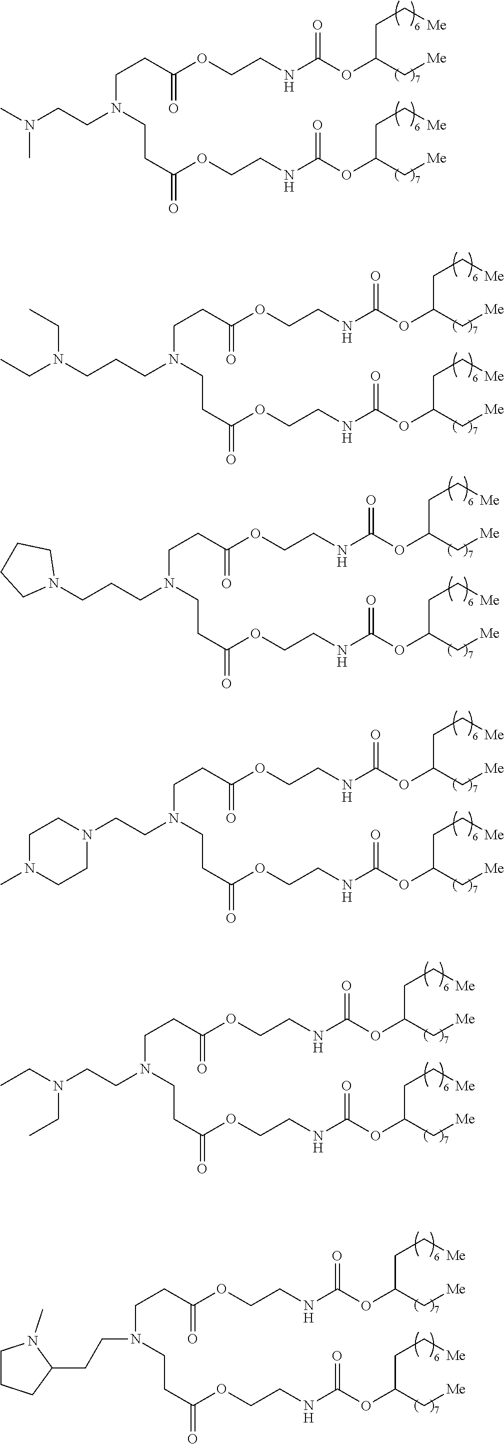

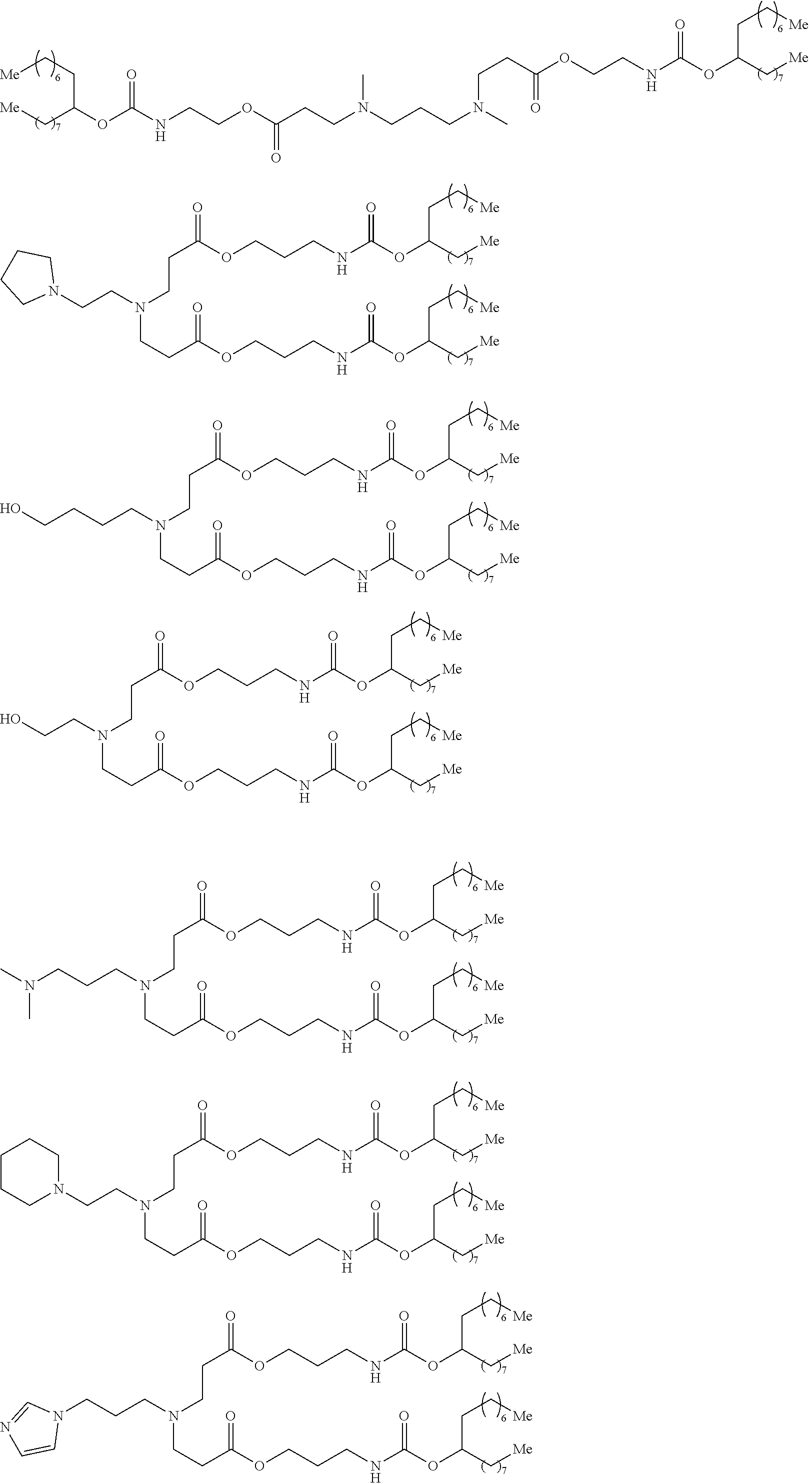

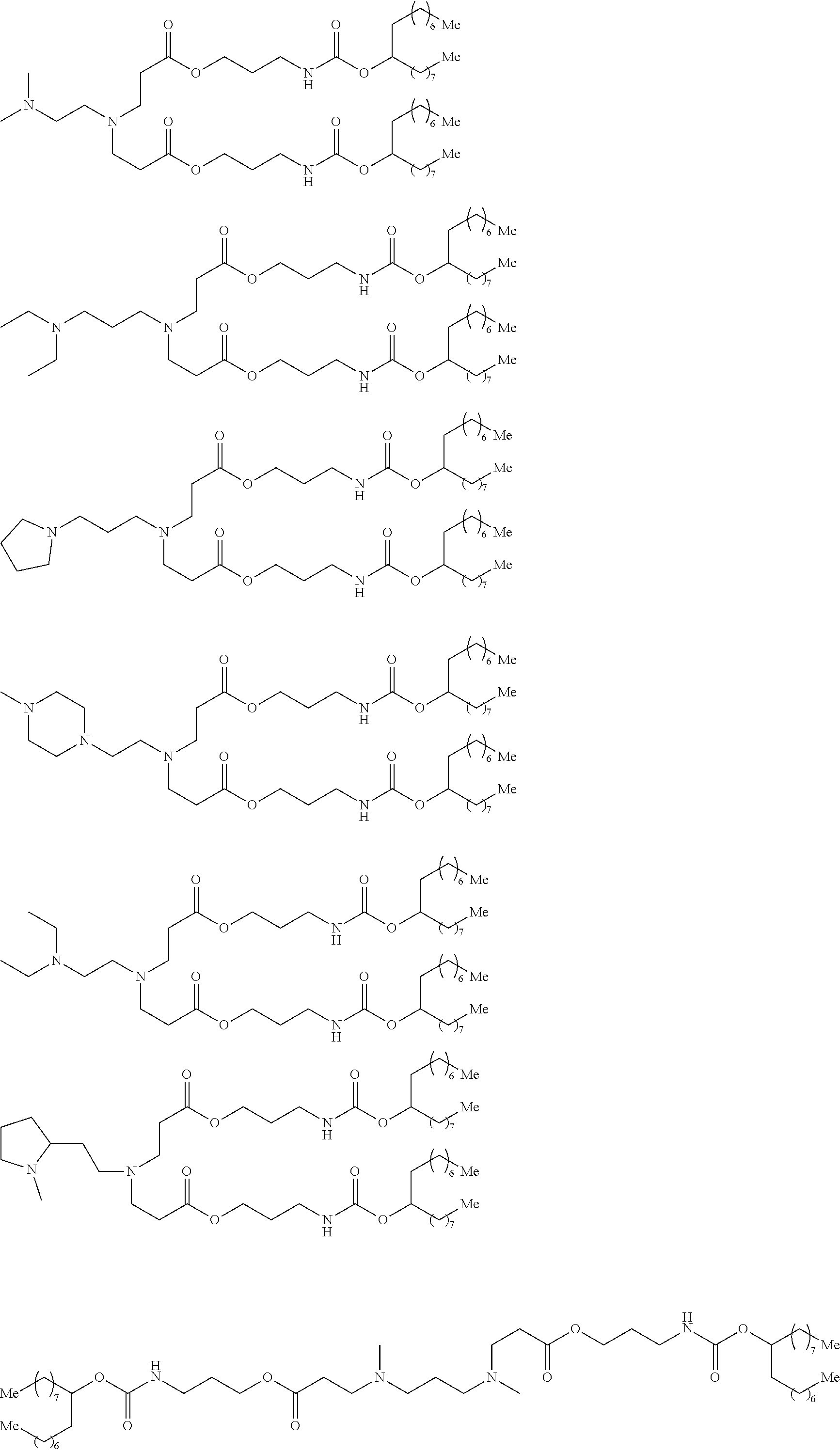

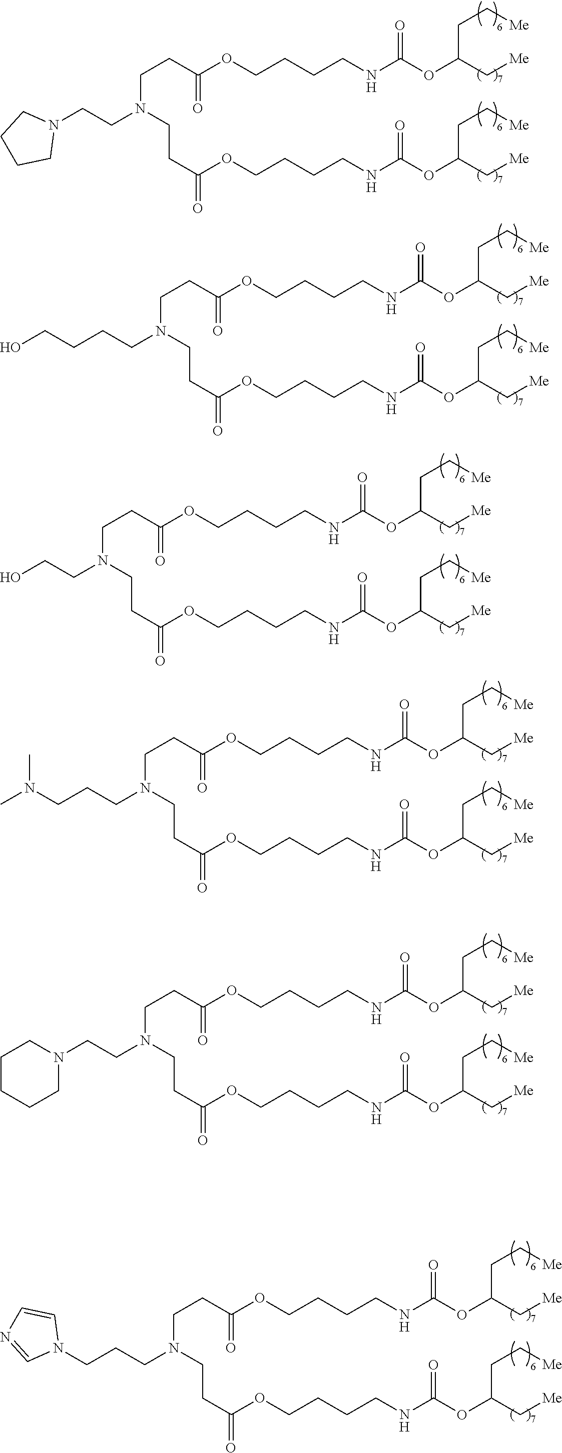

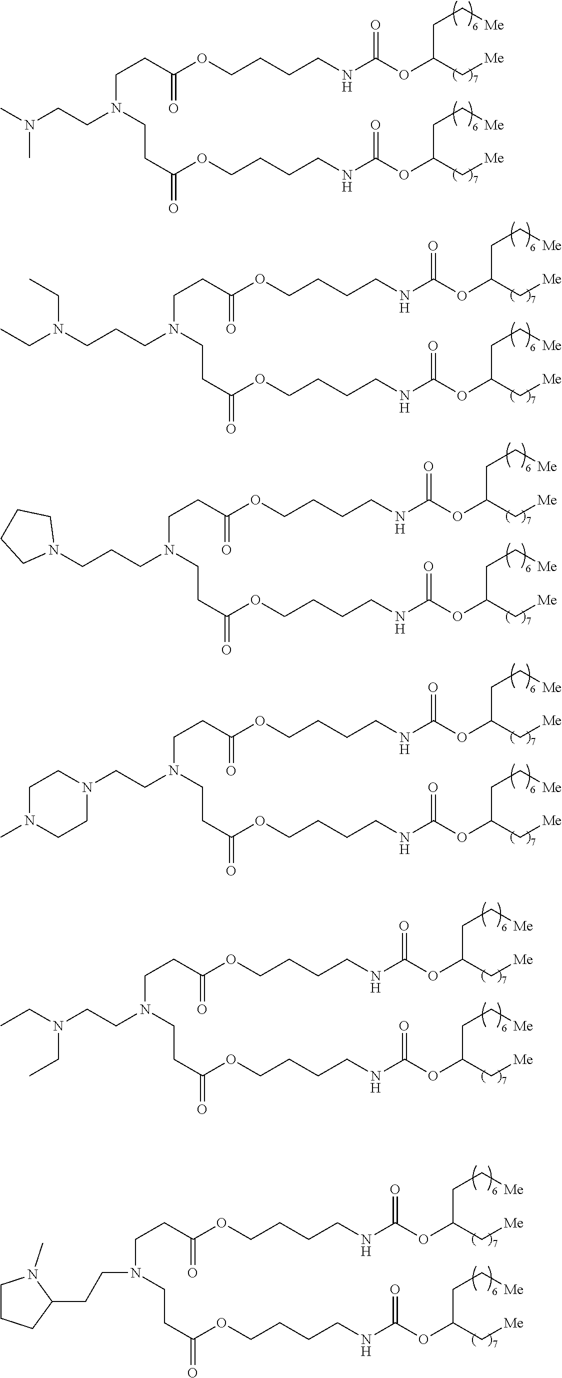

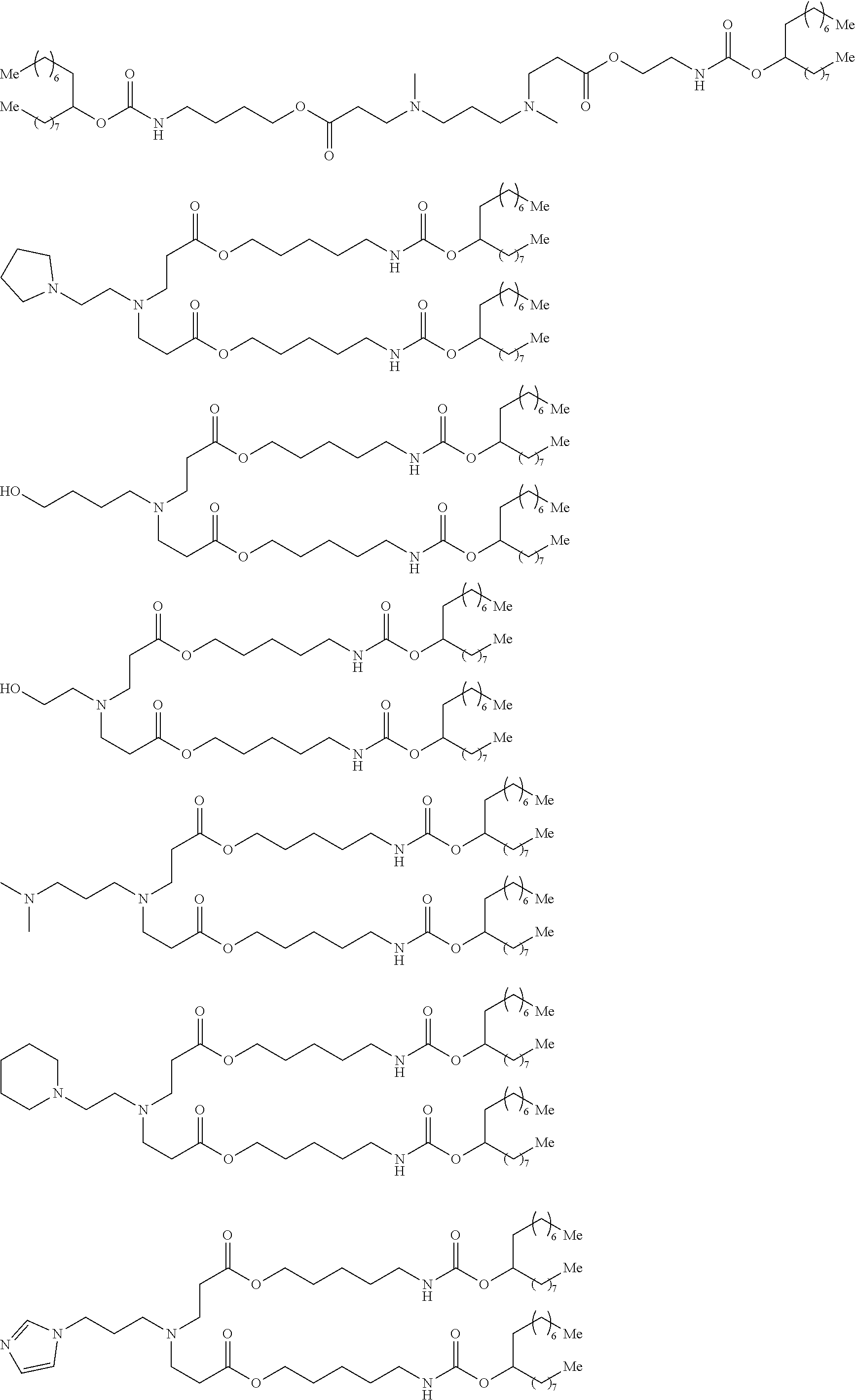

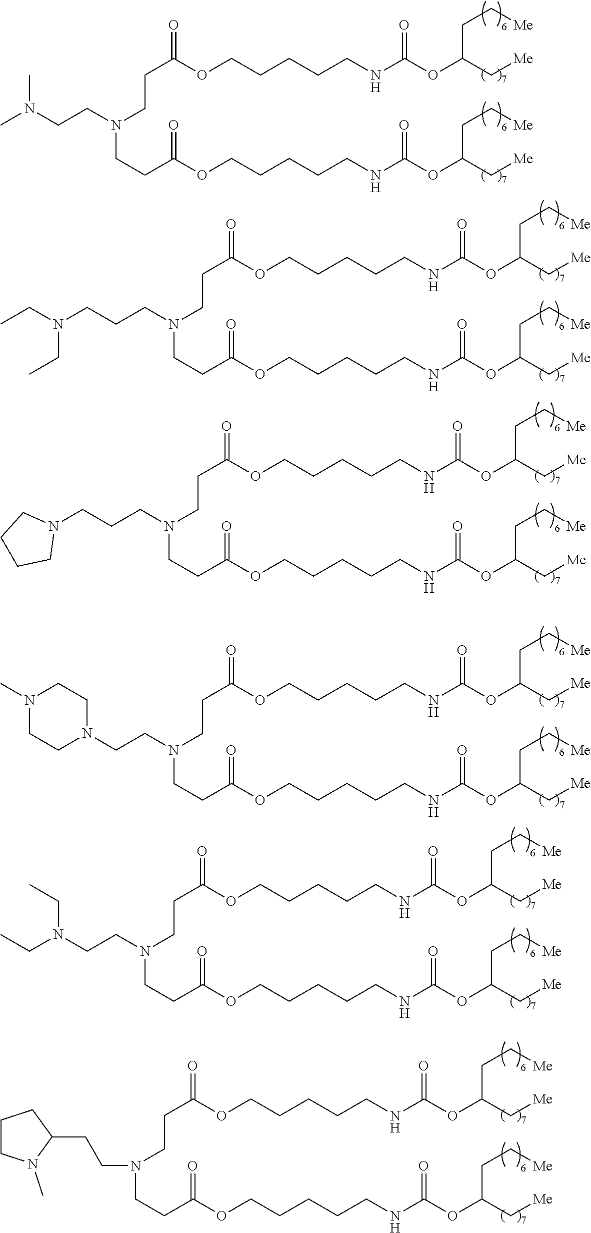

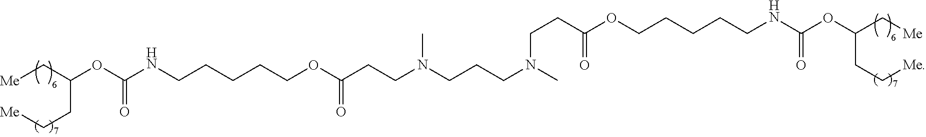

19. The method according to claim 6, wherein in the Formula (I) of the lipid compound, A is selected from a structure shown in the Formula (3), the Formula (5)-the Formula (16), B is selected from the structure shown in the Formula (19), and n is 2; and
in the Formula (19), R¹ is selected from an alkyl group of C7-C9, and m is selected from a positive integer of 2-5.

20. The method according to claim 19, wherein the lipid compound comprises a compound with the following structures: 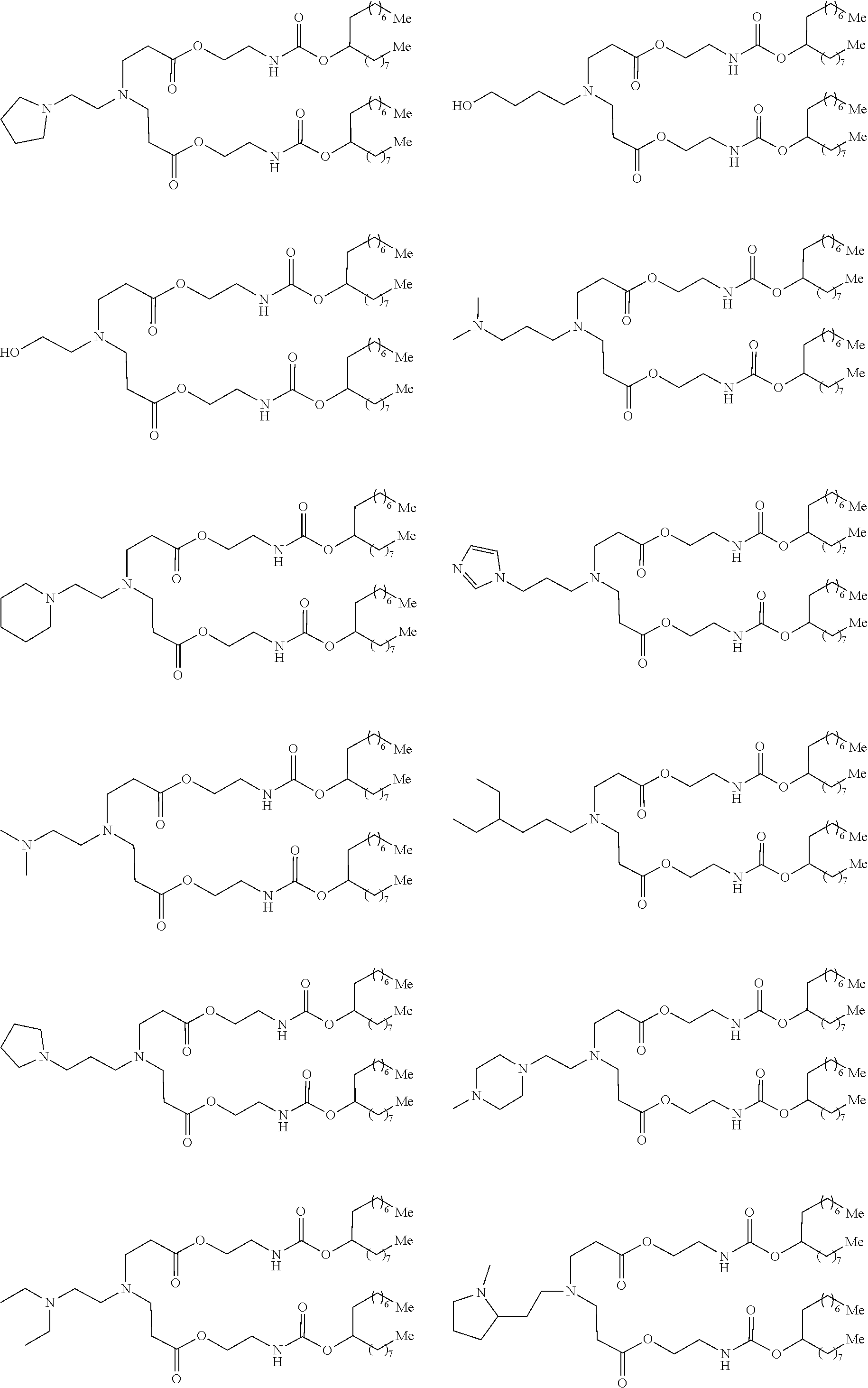 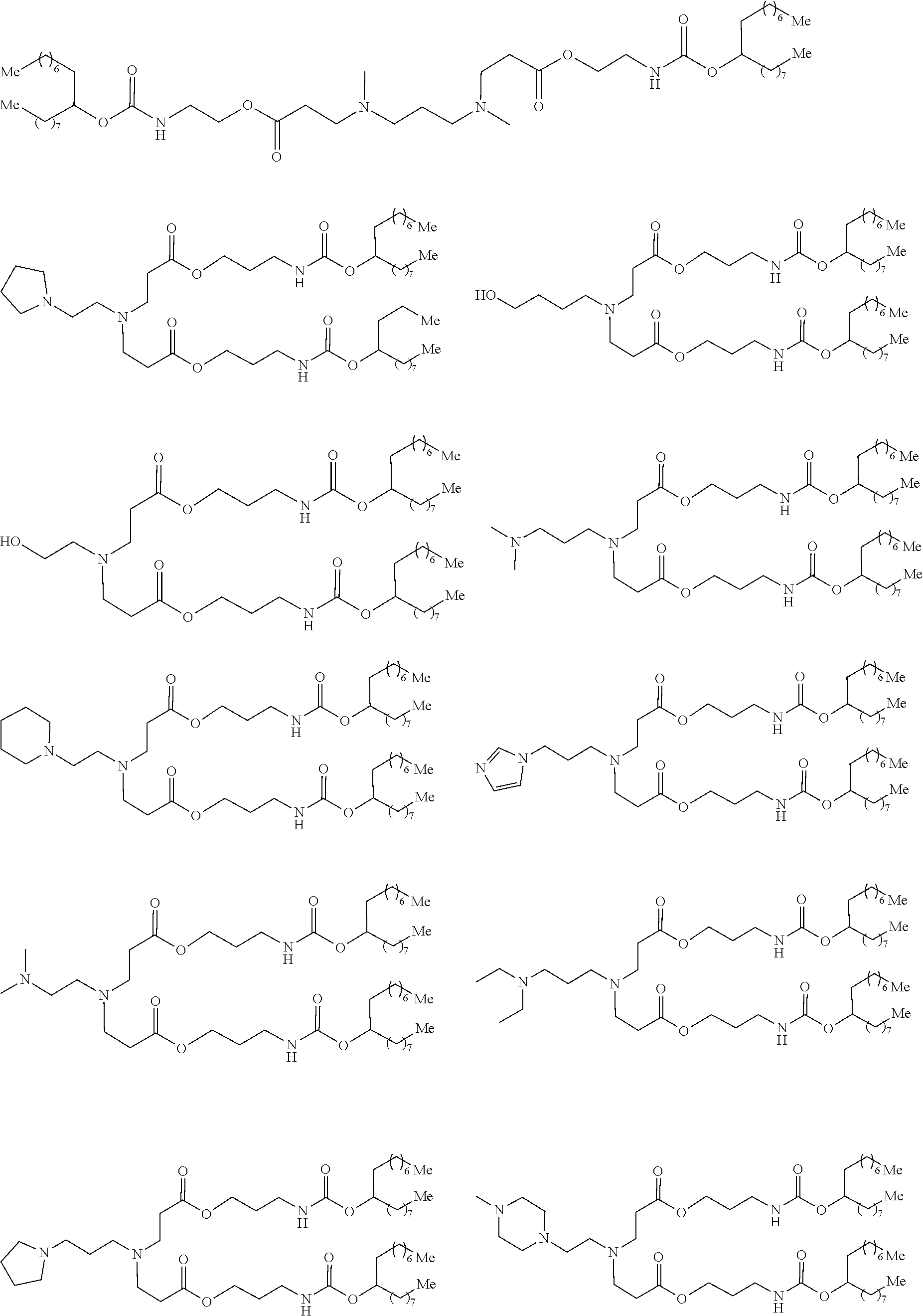 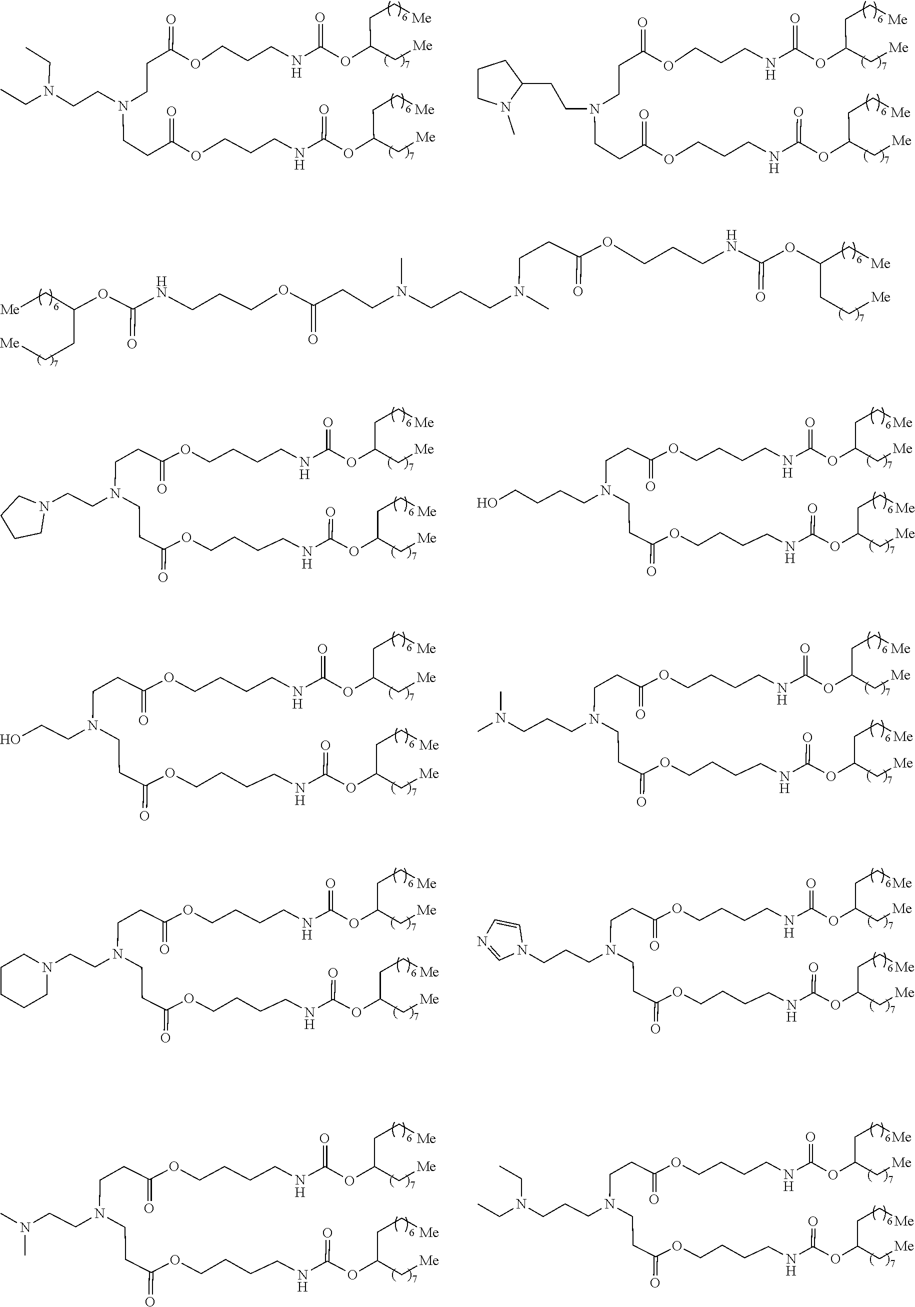 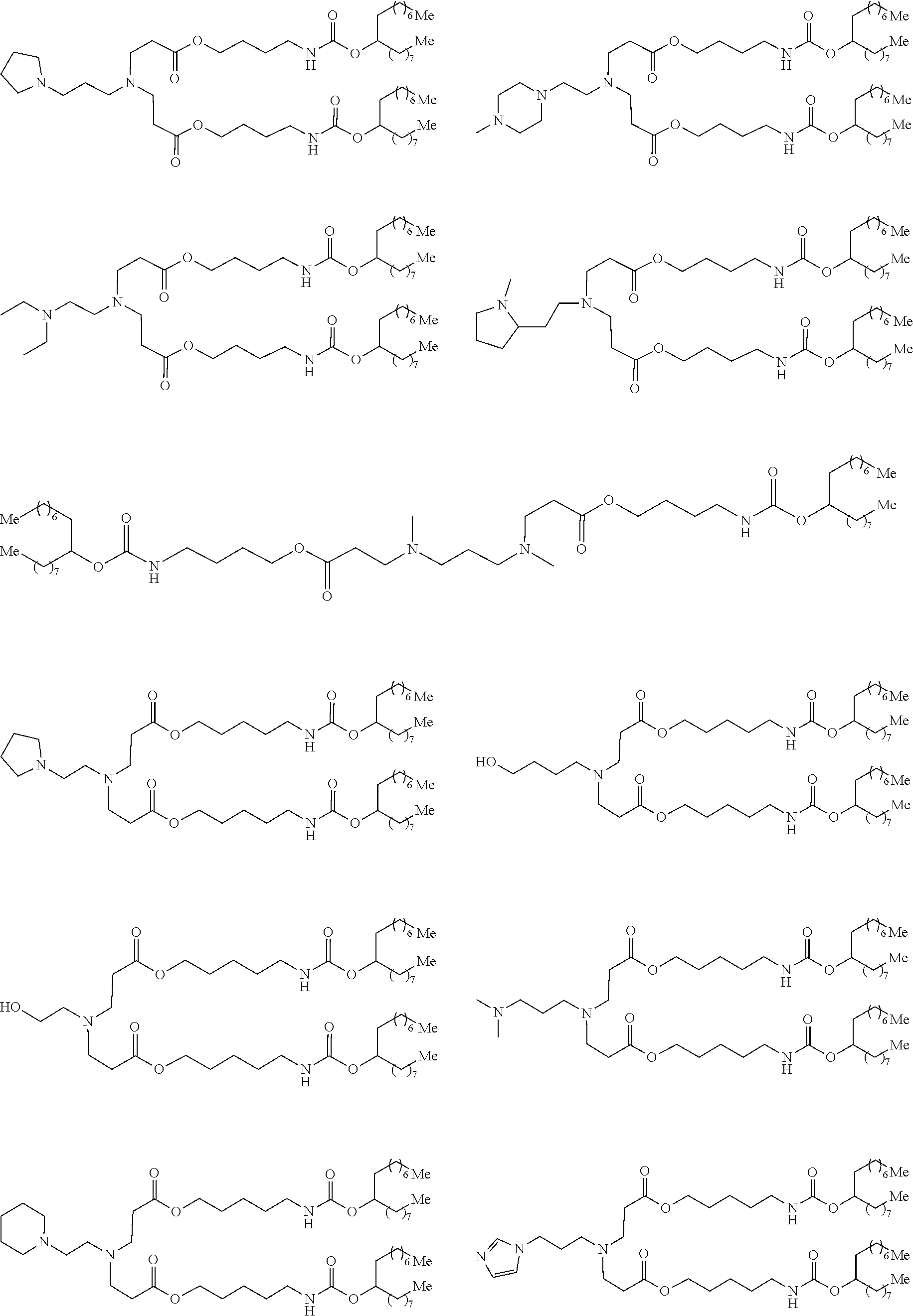 [chemical structure]
